(19) United States
(12) Patent Application Publication
(10) Pub. No.: US 2012/0316496 A1
(43) Pub. Date: Dec. 13, 2012

(54) USE OF COMPOSITIONS TO COAT CATHETER BALLOONS AND COATED CATHETER BALLOONS

(76) Inventors: Michael Hoffmann, Eschweiler (DE); Erika Hoffmann, Eschweiler (DE); Roland Horres, Stolberg (DE)

(21) Appl. No.: 13/500,875

(22) PCT Filed: Oct. 16, 2010
(86) PCT No.: PCT/DE2010/001220

§ 371 Date: Jul. 25, 2012

(30) Foreign Application Priority Data
Oct. 16, 2009 (DE) 10 2009 050 295.5

(51) Int. Cl. A61M 25/10

(52) U.S. Cl. 604/103.02

(57) ABSTRACT

The present invention is related to dilatable medical products having short-term contact with the organism, as e.g. balloon catheters coated with at least one layer of at least one antiproliferative, immunosuppressive, anti-angiogenic, anti-inflammatory, fungicidal and/or anti-thrombotic agent and a transport mediator or a mixture of transport mediators, methods for coating of these coated dilatable medical products and the use of compositions for this coating.

### BACKGROUND OF THE INVENTION

1. Field of the Invention

The present invention is related to dilatable medical products coming into short-term contact with the organism, such as balloon catheters coated with at least one layer of at least one antiproliferative, immunosuppressive, anti-angiogenic, anti-inflammatory, fungicidal and/or anti-thrombotic agent and a transport mediator or a mixture of transport mediators, methods for coating of these coated dilatable medical products and the use of compositions for this coating.

2. Description of the Relevant Art

Since the end of the 80ies of the last century metallic tubular stent grafts adjusted to the corporal lumen have been established ever the more for the prevention of restenosis, i.e. the prevention of re-occlusion of vessels, pressing when implanted from the inside against the vascular wall. Further development of these implants known as stents to a drug-coated “drug eluting stent” is currently intensively pursued due to positive results in minimizing restenosis rates in comparison with uncoated stents.

These long-term implants increasingly replaced PCTA (percutaneous transluminal coronary angioplasty) carried out since the 60ies and occupy nowadays the major part of interventions performed, since re-occlusion rates of uncoated stents are in several cases lower than occlusions recurring after PCTA performance.

The successful idea of combining mechanical and chemical prophylaxis as realised in drug eluting stents was already investigated since the very early days of stents in balloon catheters for preventing restenosis of coronary arteries and used in different varieties in clinical studies.

The drug-loaded balloon catheter, however, could not prevail over the stent. The reasons are obvious:

In PCTA the occluded part is dilated for a short time of 1 to 3 minutes by means of an inflatable balloon at the catheter tip, if necessary repeated for more than two times. Thereby the vessels have to be overstretched in such a way that the occlusion is removed. From this procedure microlesions result in the vascular walls reaching out to the adventitia. After removing the catheter the lesioned vessel is left alone so that considerably high performance is required for the healing process, depending on the inflicted lesion grade resulting from the duration, the repetitions and the grade of overstretching. This is reflected in the high re-occlusion rate after PCTA.

In stent implantation the balloon catheter is used as a transport and implant aid such that overstretching of the vascular wall occurs here as well, but in this case overstretching is only needed for the time of stent dilation. Once the stent is immovably placed in the correct position the balloon is deflated again and removed. Thus, the duration of overstretching is shorted and applied once. The reduction in restenosis rate shows that this shortened overstretching duration and the likewise reduced degree of overstretching in stents already results in a reduced rate in post-treatment, despite of introducing exogenous material into the body.

This promising advance did not leave much space for further optimizing PCTA since there was confidence that stents as permanent implants are hopeful carriers of a new preferably restenosis-free era which led to a preferential use down to the present day. PTCA is only in less severe cases and performed ahead of stent implantation in particularly severe cases for predilation of the vessel part to be treated.

The next goal in stent history is the 100% prevention of restenosis. Therefore the search for the combination of an ideal drug and an ideal preferably biodegradable stent has set out. Suppression of cellular reactions during the first days and weeks is mainly accomplished by means of preferably antiproliferative, immunosuppressive and/or antiphlogistic agents and their equally active derivatives/analogues and metabolites. The active agents and/or combinations of active agents are used herein in a sensible way for wound healing or in support of the wound healing progress.

The improvements balloon catheters have undergone in recent time were and are related so far mainly to their abilities of placing a stent precisely and safely. PCTA as an independent method has been widely replaced by stent implantation most of all in the coronary field.

But when using PCTA there are advantages over the stent, not least because at no time after performing the treatment an exogenous object is present in the organism as an additional stress factor or initiator of sequelae as is restenosis. Therefore there were and are continuations to the studies on drug-releasing balloon catheters carried out in the late 80ies.

Thus for example different embodiments of balloon catheters were described, in which the sheath being in direct contact with the environment has orifices through which a liquid or solved active agent is pressed under pressure during dilation against the vascular wall (e.g. in U.S. Pat. No. 5,087,244, U.S. Pat. No. 4,994,033, U.S. Pat. No. 4,186,745).

Here the big problem remains of providing coatings which on the one hand release sufficient active agent to the vessel wall during the short dilatation times of few minutes, normally 3 to 5 minutes, and on the other hand sufficiently stick to the active agent during the insertion of the catheter, and protect the active agent from being washed off or removed prematurely.

For example, EP 0 383 429 A discloses a balloon catheter with tiny orifices through which a heparin solution is released to the vascular wall during dilatation.

Several disadvantages such as a lower uptake of the active agent into the vascular wall, missing control on dosage, problems with the balloon material etc. kept this option of an exogenous object-free treatment of stenoses in an experimental stage. Coating of balloons analogous to stents with active agents with or without polymeric matrix also caused problems, which are on one hand originated in the short contact time and consequently a lower substance release from the catheter to its environment, and on the other hand in the considerable difficulties to bring the coating on the balloon unscathed to its destination, before and during dilation.

Only recently a substance releasing balloon catheter became an alternative to stents (CardioNews Letter, 04-21-2006). It involves a balloon catheter dipped into a solution of paclitaxel and a radiocontrast medium which led, according to the results of a one year clinical study, to a reduction in restenosis rate from 40 to 9% as compared to an uncoated balloon catheter. For example, such a balloon catheter is disclosed in WO 2004028582 A1.

Though even these first results seem to be promising, typical problems of such a treatment have not been overcome.

In any case the optical traceability achieved by the coating with a contrast medium is advantageous, but the amount of the active agent effectively released and taken up at the site of action after PTCA performance remains individual and uncontrolled, since an unquantifiable portion of the coating is released already during introducing the balloon catheter into the bloodstream starting from the groin to the heart. Additionally, also during balloon dilation further parts of the coating crumble off and are carried away from the surface by the bloodstream. Consequently, a part of the concentration of the active agent applied to the balloon catheter does not reach the affected site, but can be simply regarded as an ineffective intravenous administration. The amount of the lost portion cannot be controlled and is thus not available for an optimal treatment at the affected site and in a controllable dose. What remains on the balloon catheter has therefore to be sufficient for achieving a promising therapy, but likewise the question remains how much of the active agent actually reaches its target and is actually absorbed by the vascular wall, and whether this amount is sufficient to achieve the desired success.

Thus the possibility of a stent free restenosis treatment shown by this balloon catheter shall be brought on new, effective and controllable roads.

Further, the conventional method of dip as well as spray coating for catheter balloons has the great disadvantage that it can never be exactly determined how much substance actually was applied to the balloon surface which leads to a situation where basically a significant overdose occurs. Moreover it becomes ever the more important in terms of regulatory affairs and for attaining marketing authorizations to provide well defined balloon coatings for which the substance amount was exactly determined. Conventional methods of dipping the catheter balloon several times in a coating solution or of exposing the balloon to a spray stream or mist of the coating solution do not yield reproducible results, so that the application of a defined substance amount was not possible. Consequently, dipping methods are the worst alternative for coating catheter balloons.

### SUMMARY OF THE INVENTION

The objective of the present invention consists in providing compositions for the coating of catheter balloons which make sure a sufficient adhesion of the active agent to the balloon surface during introduction of the catheter balloon and on the other hand an optimal transfer of the active agent to the vascular wall during dilation.

Further it is the objective of the present invention to provide coating methods for the coating of catheter balloons, wherein the amount of applied coating and thereby the amount of applied active agent can be exactly estimated.

A further objective of the present invention is to provide an agent releasing balloon catheter and similar medical products for short-term use in the body which ensure a controlled and optimal agent transfer to and into the vascular wall even during short term exposure such that the healing process proceeds positively.

For this purpose it must be ensured that on one hand the active agent is not washed off from the medical product by body fluid on its way to the target site or is crumbled off at the latest upon expansion and merely an undefined respectively insufficient amount of active agent reaches the target. On the other hand the strongly limited exposure time must be sufficient to get the active agent transferred in a determined dosage from the balloon catheter onto respectively into the vascular wall.

This objective is solved by the teaching of the independent claims of the present invention. Further advantageous embodiments of the invention result from the dependent claims, the description and the examples.

### BRIEF DESCRIPTION OF THE DRAWINGS

Advantages of the present invention will become apparent to those skilled in the art with the benefit of the following detailed description of embodiments and upon reference to the accompanying drawings in which:

FIG. 1 shows a coating device according to the ballpoint method, wherein the coating solution is placed in the inside of the coating device which is released via a rotating ball onto the surface to be coated.

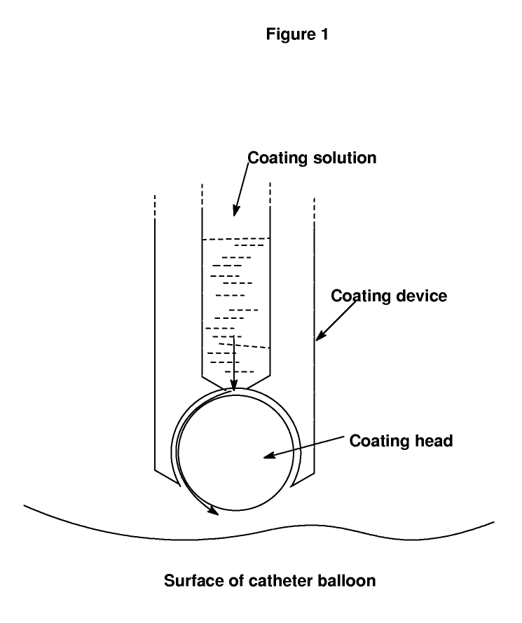

While the invention may be susceptible to various modifications and alternative forms, specific embodiments thereof are shown by way of example in the drawings and will herein be described in detail. The drawings may not be to scale. It should be understood, however, that the drawings and detailed description thereto are not intended to limit the invention to the particular form disclosed, but to the contrary, the intention is to cover all modifications, equivalents, and alternatives falling within the spirit and scope of the present invention as defined by the appended claims.

### DETAILED DESCRIPTION OF THE PREFERRED EMBODIMENTS

According to the present invention one objective is solved by special coating methods for catheter balloons coating the catheter balloon with a defined amount of a pharmacologically active agent, wherein the coating method uses a coating device with a volume measuring equipment for releasing a measurable amount of a coating solution by means of a release device specifically on the surface of the catheter balloon.

As a volume measuring equipment any device can be used which is able to provide a measured amount of coating solution or to measure or to display the amount of released coating solution. Volume measuring equipments are in the most simple case scales, scaled pipettes, scaled burettes, scaled containers, scaled cavities as well as pumps, valves, syringes or other piston-shaped containers able to provide, to transport or to release a measured amount of coating solution. Thus the volume measuring equipment only serves either to provide or to release a certain amount of coating solution or to measure and/or display a released amount of coating solution. Thus the volume measuring equipment serves to determine or rather to measure the amount of coating solution transferred from the release device to the surface of the catheter balloon and thereby the amount of agent.

The object in regard to sufficient adhesion and release of active agent upon dilation is solved by a specific coating solution containing a preferred transport mediator or preferred mixtures of transport mediators. The transport mediators are referred to in more detail further below in a separated paragraph. The use of the term “transport mediator” stands in the following for a single transport mediator as well as for a mixture of transport mediators.

The key component of the coating device is, however, the release device which can be realized as a nozzle, a plurality of nozzles, a thread, a mesh of threads, a piece of textile, a leather strip, a sponge, a ball, a syringe, a needle, a cannula or a capillary. Depending on the embodiment of the release device slightly modified coating methods result which are all based on the principle of transferring a measurable or predetermined but known amount of active agent on the surface of the catheter balloon, thus yielding a coating with a defined active agent concentration or amount and providing reproducible coatings having little deviations with respect to each other, which conventional dip or spray methods are not able to serve. For differentiating the methods different terms are used herein, namely squirting method, pipetting method, capillary method, fold spray method, drag method, thread drag method or roll method, which are the preferred embodiments of the present invention.

Not only a particular method but also a particular device result from the use of a ball as releasing device. The corresponding method is described herein as roll method and the corresponding device has a ball head with a supply for the coating solution to the ball head. By means of a control, preferably an optical control, the ball head is put onto the surface of the catheter balloon. Through a valve or due to the pressure of the balloon surface on the ball head the coating solution flows out of a cavity or a volume measuring equipment onto the ball head. The ball head is rolled over the surface of the catheter balloon and thereby drives off the surface of the catheter balloon, wherein the coating solution added to the ball head is transferred from the ball head to the surface of the catheter balloon.

By means of such a device and with this roll method catheter balloons can be coated completely or only partially in the deflated or inflated state. For example, a catheter balloon can be specifically driven off and coated in the inflated or partially inflated state in the region of the widened folds, wherein the coating remains onside the folds after deflation (i.e. folding up), such that a specific coating of the folds can be achieved that way. In order to avoid that the ball damages the balloon or rather the balloon material this material is preferably made of a rubber-like material like e.g. natural rubber or at least one other polymer suitable for this object.

The single preferred coating methods are referred to in detail further below.

The present invention refers particularly to coated catheter balloons with an agent releasing coating.

As catheter balloons conventional catheter balloons, bifurcation balloons as well as fold balloons or special balloons can be used.

The term “catheter balloons” or “conventional catheter balloons” refers to such dilatable catheter balloons which usually serve to place a stent by means of dilation. Further, it refers also to non-dilatable catheter balloons for stent placement which are suitable for self-expanding stents and carry a removable sheath on the stent for avoiding premature stent expansion.

Expandable and re-compressible catheter balloons with a sheath as in non-dilatable catheter balloons for self-expanding stents are, however, usually used without a stent in order to protect the coating on the catheter balloon from premature removal.

Bifurcation balloons refer to catheter balloons for treating a bifurcation of a vessel, especially of a blood vessel. Such balloons may have two arms or consist of two combined or two separate balloons being used simultaneously or consecutively for the treatment of a vessel bifurcation or rather the placement of one or two stents in a vessel bifurcation or in the direct proximity to a vessel bifurcation.

“Fold balloons” refer to balloons as described for example in EP 1189553 B1, EP 0519063 B1, WO 03/059430 A1 and WO 94/23787 A1, having “folds” in the compressed state of the balloon that open at least partially when expanding the balloon. Usually every balloon used for angioplasty can be referred to as fold balloon because all such balloons have folds in the deflated state.

Special balloons refer to balloons with pores, particularly with micropores, allowing liquids and solutions to pass through during expansion or upon application of pressure. Such a balloon with micropores is disclosed in EP 0 383 429 A. Further, the term “special balloon” refers to balloons with a specifically designed surface as for example the one catheter balloon with microneedles described in WO 02/043796 A2 or the one catheter balloon with a micro raw or nano raw surface for embedding active agents with or without carrier substances disclosed in WO 03/026718 A1.

The term “balloon” or “catheter balloon” basically refers to every expandable and re-compressible as well as to temporarily implantable medical device which are usually used together with a catheter.

The inventive coated balloons can be used without a stent or with a crimped stent. Their use is not only limited to a first treatment of stenotic vessels but they are also particularly well suitable to combat successfully an occurring restenosis (e.g. in-stent-restenosis) and to prevent recurrent re-occlusion.

The catheter balloon can consist of the common materials, especially polymers as described further below, and particularly of polyamide such as PA 12, polyester, polyurethane, polyacrylates, polyethers and so on.

The stent may consist likewise of the common materials such as for example medical stainless steel, titanium, chromium, vanadium, tungsten, molybdenum, gold, iron, nitinol, magnesium, iron, alloys of aforementioned metals as well as polymeric material and preferably resorbable polymeric materials such as chitosan and its derivatives, poly amino acids, polypeptides, polyhydroxybutyrates (PHB), polyvinylpyrrolidone, polyvinyl alcohols, polyglyceroles, polylactides and the block- and copolymers of the aforementioned materials.

The inventive coated catheter balloons are preferably used without an attached stent, but a use with a crimped stent is also possible. Using besides the coated balloon a stent crimped thereon the stent may then be uncoated (bare stent) or likewise coated, wherein the stent may have a different coating and also a different active agent than the coating of the catheter balloon.

The term “coating” shall comprise not only a coating of the surface of the catheter balloon but also a filling or coating of folds, cavities, pores, microneedles or other fillable spaces on or between or in the balloon material.

The coating may be applied in one or several steps, may have one or more layers, wherein the coating solution contains a suitable solvent or mixture of solvents, one or also several pharmacologically active agents, and a transport mediator or a mixture of transport mediators. Further components can be contained in the coating solution, wherein usually the coating solution preferably only consists of the three aforementioned components. Suitable active agents or combinations of active agents are anti-inflammatory, cystostatic, cytotoxic, antiproliferative, anti-microtubuli, anti-angiogenic, anti-restenotic (anti-restenosis), fungicide, antineoplastic, antimigrative, athrombogenic and antithrombogenic substances.

As anti-inflammatory, cystostatic, cytotoxic, antiproliferative, anti-microtubuli, anti-angiogenic, anti-restenotic, fungicide, antineoplastic, antimigrative, athrombogenic and antithrombogenic substances can preferably be used: vasodilators, sirolimus (rapamycin), somatostatin, tacrolimus, roxithromycin, dunaimycin, ascomycin, bafilomycin, erythromycin, midecamycin, josamycin, concanamycin, clarithromycin, troleandomycin, folimycin, cerivastatin, simvastatin, lovastatin, fluvastatin, rosuvastatin, atorvastatin, pravastatin, pitavastatin, vinblastine, vincristine, vindesine, vinorelbine, etoposide, teniposide, nimustine, carmustine, lomustine, cyclophosphamide, 4-hydroxycyclophosphamide, estramustine, melphalan, ifosfamide, trofosfamide, chlorambucil, bendamustine, dacarbazine, busulfan, procarbazine, treosulfan, temozolomide, thiotepa, daunorubicin, doxorubicin, aclarubicin, epirubicin, mitoxantrone, idarubicin, bleomycin, mitomycin, dactinomycin, methotrexate, fludarabine, fludarabine-5′-dihydrogenephosphate, cladribine, mercaptopurine, thioguanine, cytarabine, fluorouracil, gemcitabine, capecitabine, docetaxel, carboplatin, cisplatin, oxaliplatin, amsacrine, irinotecan, topotecan, hydroxycarbamide, miltefosine, pentostatin, aldesleukin, tretinoin, asparaginase, pegaspargase, anastrozole, exemestane, letrozole, formestane, aminoglutethimide, adriamycin, azithromycin, spiramycin, cepharantin, 8-α-ergoline, dimethylergoline, agroclavin, 1-allylisurid, 1-allyltergurid, bromergurid, bromocriptin (ergotaman-3′,6′,18-trione, 2-bromo-12′-hydroxy-2′-(1-methylethyl)-5′-(2-methylpropyl)-, (5′ alpha)-), elymoclavin, ergocristin (ergotaman-3′,6′,18-trione, 12′-hydroxy-2′-(1-methylethyl)-5′-(phenylmethyl)-, (5′-alpha)-), ergocristinin, ergocornin (ergotaman-3′,6′,18-trione, 12′-hydroxy-2′,5′-bis(1-methylethyl)-, (5′-alpha)-), ergocorninin, ergocryptin (ergotaman-3′,6′,18-trione, 12′-hydroxy-2′-(1-methylethyl)-5′-(2-methylpropyl)-, (5′ alpha)-(9CI)), ergocryptinin, ergometrin, ergonovin (ergobasin, INN: ergometrin, (8beta(S))-9,10-didehydro-N-(2-hydroxy-1-methylethyl)-6-methyl-ergoline-8-carboxamid), ergosin, ergosinin, ergotmetrinin, ergotamin (ergotaman-3′,6′,18-trione, 12′-hydroxy-2′-methyl-5′-(phenylmethyl)-, (5′-alpha)-(9CI)), ergotaminin, ergovalin (ergotaman-3′,6′,18-trione, 12′-hydroxy-2′-methyl-5′-(1-methylethyl)-, (5′ alpha)-), lergotril, lisurid (CAS-No.: 18016-80-3,3-(9,10-didehydro-6-methylergolin-8alpha-yl)-1,1-diethyl carbamide), lysergol, lysergic acid (D-lysergic acid), lysergic acid amide (LSA, D-lysergic acid amide), lysergic acid diethylamide (LSD, D-lysergic acid diethylamide, INN: lysergamide, (8β)-9,10-didehydro-N,N-diethyl-6-methyl-ergoline-8-carboxamide), isolysergic acid (D-isolysergic acid), isolysergic acid amide (D-isolysergic acid amide), isolysergic acid diethylamide (D-isolysergic acid diethylamide), mesulergin, metergolin, methergin (INN: methylergometrin, (8beta(S))-9,10-didehydro-N-(1-(hydroxymethyl)propyl)-6-methyl-ergoline-8-carboxamide), methylergometrin, methysergid (INN: methysergid, (8beta)-9,10-didehydro-N-(1-(hydroxymethyl)propyl)-1,6-dimethyl-ergoline-8-carboxamide), pergolid ((8β)-8-((methylthio)methyl)-6-propyl-ergolin), protergurid and tergurid, celecoxip, thalidomid, Fasudil®, ciclosporin, smc proliferation inhibitor-2w, epothilone A and B, mitoxantrone, azathioprine, mycophenolatmofetil, c-myc-antisense, b-myc-antisense, betulinic acid, camptothecin, PI-88 (sulfated oligosaccharide), melanocyte-stimulating hormone (α-MSH), activated protein C, thymosine α-1, fumaric acid and its esters, calcipotriol, tacalcitol, lapachol, β-lapachone, podophyllotoxin, betulin, podophyllic acid 2-ethylhydrazide, molgramostim (rhuGM-CSF), peginterferon α-2b, lanograstim (r-HuG-CSF), filgrastim, macrogol, dacarbazin, basiliximab, daclizumab, selectin (cytokine antagonist), CETP inhibitor, cadherines, cytokinin inhibitors, COX-2 inhibitor, NFkB, angiopeptin, ciprofloxacin, camptothecin, fluoroblastin, monoclonal antibodies which inhibit the muscle cell proliferation, bFGF antagonists, probucol, prostaglandins, 1,11-dimethoxycanthin-6-on, 1-hydroxy-11-methoxycanthin-6-on, scopolectin, colchicine, NO donors such as pentaerythritol tetranitrate and syndnoeimines, S-nitrosoderivatives, tamoxifen, staurosporine, β-estradiol, α-estradiol, estriol, estrone, ethinylestradiol, fosfestrol, medroxyprogesterone, estradiol cypionates, estradiol benzoates, tranilast, kamebakaurin and other terpenoids which are applied in the therapy of cancer, verapamil, tyrosine kinase inhibitors (tyrphostines), cyclosporine A and B, paclitaxel and its derivatives such as 6-α-hydroxy-paclitaxel, baccatin, taxotere, synthetically produced macrocyclic oligomers of carbon suboxide (MCS) and its derivatives as well as those obtained from native sources, mofebutazone, acemetacin, diclofenac, lonazolac, dapsone, o-carbamoylphenoxyacetic acid, lidocaine, ketoprofen, mefenamic acid, piroxicam, meloxicam, chloroquine phosphate, penicillamine, tumstatin, avastin, D-24851, SC-58125, hydroxychloroquine, auranofin, sodium aurothiomalate, oxaceprol, celecoxib, β-sitosterin, ademetionine, myrtecaine, polidocanol, nonivamide, levomenthol, benzocaine, aescin, ellipticine, D-24851 (Calbiochem), colcemid, cytochalasin A-E, indanocine, nocodazole, S 100 protein, bacitracin, vitronectin receptor antagonists, azelastine, guanidyl cyclase stimulator, tissue inhibitor of metal proteinase-1 and -2, free nucleic acids, nucleic acids incorporated into virus transmitters, DNA and RNA fragments, plasminogen activator inhibitor-1, plasminogen activator inhibitor-2, antisense oligonucleotides, VEGF inhibitors, IGF-1, active agents from the group of antibiotics such as cefadroxil, cefazolin, cefaclor, cefotixin, tobramycin, gentamycin, penicillins such as dicloxacillin, oxacillin, sulfonamides, metronidazol, antithrombotics such as argatroban, aspirin, abciximab, synthetic antithrombin, bivalirudin, coumadin, enoxaparin, desulfated and N-reacetylated heparin, tissue plasminogen activator, GpIIb/IIIa platelet membrane receptor, factor Xₐ inhibitor antibodies, interleukin inhibitors, heparin, hirudin, r-hirudin, PPACK, protamine, sodium salt of 2-methylthiazolidin-2,4-dicarboxylic acid, prourokinase, streptokinase, warfarin, urokinase, vasodilators such as dipyramidole, trapidil, nitroprus sides, PDGF antagonists such as triazolopyrimidine and seramin, ACE inhibitors such as captopril, cilazapril, lisinopril, enalapril, losartan, thioprotease inhibitors, prostacyclin, vapiprost, interferon α, β and γ, histamine antagonists, serotonin blockers, apoptosis inhibitors, apoptosis regulators such as p65, NF-kB or Bcl-xL antisense oligonucleotides, halofuginone, nifedipine, tocopherol, vitamin B1, B2, B6 and B12, folic acid, tranilast, molsidomine, tea polyphenols, epicatechin gallate, epigallocatechin gallate, Boswellic acids and their derivatives, leflunomide, anakinra, etanercept, sulfasalazine, etoposide, dicloxacillin, tetracycline, triamcinolone, mutamycin, procainamid, D24851, SC-58125, retinoic acid, quinidine, disopyramide, flecamide, propafenone, sotalol, amidorone, natural and synthetically prepared steroids such as bryophyllin A, inotodiol, maquirosid A, ghalakinosid, mansonin, streblosid, hydrocortisone, betamethasone, dexamethasone, non-steroidal substances (NSAIDS) such as fenoprofen, ibuprofen, indomethacin, naproxen, phenylbutazone and other antiviral agents such as acyclovir, ganciclovir and zidovudine, antimycotics such as clotrimazole, flucytosine, griseofulvin, ketoconazole, miconazole, nystatin, terbinafine, antiprotozoal agents such as chloroquine, mefloquine, quinine, furthermore natural terpenoids such as hippocaesculin, barringtogenol-C21-angelate, 14-dehydroagrostistachin, agroskerin, agrostistachin, 17-hydroxyagrostistachin, ovatodiolids, 4,7-oxycycloanisomelic acid, baccharinoids B1, B2, B3 and B7, tubeimoside, bruceanol A, B and C, bruceantinoside C, yadanziosides N and P, isodeoxyelephantopin, tomenphantopin A and B, coronarin A, B, C and D, ursolic acid, hyptatic acid A, zeorin, iso-iridogermanal, maytenfoliol, effusantin A, excisanin A and B, longikaurin B, sculponeatin C, kamebaunin, leukamenin A and B, 13,18-dehydro-6-α-senecioyloxychaparrin, taxamairin A and B, regenilol, triptolide, furthermore cymarin, apocymarin, aristolochic acid, anopterin, hydroxyanopterin, anemonin, protoanemonin, berberine, cheliburin chloride, cictoxin, sinococuline, bombrestatin A and B, cudraisoflavone A, curcumin, dihydronitidine, nitidine chloride, 12-beta-hydroxypregnadiene-3,20-dione, bilobol, ginkgol, ginkgolic acid, helenalin, indicine, indicine-N-oxide, lasiocarpine, inotodiol, glycoside 1a, podophyllotoxin, justicidin A and B, larreatin, malloterin, mallotochromanol, isobutyrylmallotochromanol, maquiroside A, marchantin A, maytansine, lycoridicin, margetine, pancratistatin, liriodenine, oxoushinsunine, aristolactam-AII, bisparthenolidine, periplocoside A, ghalakinoside, ursolic acid, deoxypsorospermin, psychorubin, ricin A, sanguinarine, manwu wheat acid, methylsorbifolin, sphatheliachromen, stizophyllin, mansonine, strebloside, akagerine, dihydrousambarensine, hydroxyusambarine, strychnopentamine, strychnophylline, usambarine, usambarensine, berberine, liriodenine, oxoushinsunine, daphnoretin, lariciresinol, methoxylariciresinol, syringaresinol, umbelliferon, afromoson, acetylvismione B, desacetylvismione A, vismione A and B, and sulfur-containing amino acids such as cysteine as well as salts, hydrates, solvates, enantiomers, racemates, enantiomeric mixtures, diastereomeric mixtures, metabolites, prodrugs and mixtures of the aforementioned active agents.

Basically all active agents as well as combinations of active agents can be used, wherein, however, paclitaxel and paclitaxel derivatives, taxanes, docetaxel as well as rapamycin and rapamycin derivatives such as biolimus A9, pimecrolimus, everolimus, zotarolimus, tacrolimus, fasudil and epothilones are preferred, and particularly preferred are paclitaxel and rapamycin.

Paclitaxel is known under the brand name Taxol® and the chemical name [2aR-[2a,4,4a,6,9 (R*,S*),11,12,12a,12b]]-(benzoylamino)-hydroxybenzolpropionic acid-6,12b-bis-(acetyloxy)-12-(benzoyloxy)-2a-3,4,4a,5,6,9,10,11,12,12a,12b-dodecahydro-4,11-dihydroxy-4a,8,13,13-tetramethyl-5-oxo-7,11-methano-1H-cyclodeca[3,4]benz[1,2-b]oxet-9-yl ester.

Rapamycin is also known as Rapamun or under the International Nonproprietary Name (INN) sirolimus as well as under the IUPAC name [3S-[3R*[E(1S*,3S*,4S*)],4S*,5R*,8S*,9E,12R*,14R*,15S*,16R*,18S*,19S*,26aR*]]-5,6,8,11,12,13,14,15,16,17,18,19,24,25,26,26a-hexadeca-hydro-5,19-dihydroxy-3-[2-(4-hydroxy-3-methoxycyclohexyl)-1-methylethenyl]-14,16-dimethoxy-4,10,12,18-tetramethyl-8-(2-propenyl)-15,19-epoxy-3H-pyrido[2,1-c][1,4]-oxaazacyclo-tricosene-1,7,20,21(4H,23H)-tetrone monohydrate.

“Prodrugs” refers to a precursor of a pharmacologically active compound which is converted into the active compound under physiological conditions.

These active agents or combinations of active agents reach their target site preferably by means of a transport mediator or as their own transport mediator in a sufficient concentration during the limited exposure time of the short-term implant.

As already mentioned, a major problem of the embodiments of the state-of-the-art consists in transferring a sufficient amount of active agent onto the stenotic or restenotic or thrombotic vessel section within a single dilation duration of at most 1 minute and possibly several repetitions of the dilation after a certain interruption and preferably of at most 45 seconds and particularly preferably of at most 30 seconds such that a restenosis or re-occlusion of the vessel section is prevented even in a dilation without stent placement. Since the risk of a heart attack increases with higher exposure times, i.e. dilation durations, there is only little time for the transfer of the active agent(s) onto or into the vascular wall, respectively. Furthermore, a repeated expansion and recompression of the catheter balloon for ensuring temporarily at least a slight bloodstream also is critical for the so called “biological stenting” without a stent also since the active agent is released in its major part already during the first expansion of the catheter balloon and further dilations cannot contribute anymore to a considerable substance transfer onto the vascular wall.

Thus special coatings are needed which transfer in a relatively short time a relative high amount of active agent in a controlled manner onto and/or into the vascular wall.

Therefore for the preparation of these coatings on the balloon surface coating solutions are used which contain the active agent together with at least one transport mediator in a suitable and after the coating removable solvent.

### Transport Mediators

In order to increase the transfer of active agent preferably so-called transport mediators or transport accelerators are used.

These substances can additionally have active agent properties themselves or may also function as suitable carrier material for an active agent.

Of special interest are the inventive embodiments containing chemical compounds as transport mediators which accelerate or facilitate the uptake of the active agent into the vascular wall, respectively so that the present active agent or combination of active agents can be transported during the short exposure time in a controlled manner and in the predetermined dosage through the cell membrane into the inner cell.

Now, the use of transport mediators itself is not subject of the present invention, but rather the selection of particularly preferred transport mediators which are particularly well effective, especially in connection with catheter balloons and with the special needs upon the short-time active agent release, i.e. in the light of these special needs they promote the transfer of the active agent into the vascular wall and the uptake of active agent into the tissue such as the stenotic tissue, respectively.

All transport mediators have the ability in common to change the thermodynamic condition of the active agent in the ideal case, so that the concentration gradient is increased and the diffusion into the cells is enhanced.

Thereby, the transport accelerator may also function as a carrier. Several options are possible here: the linkage between the active agent and the carrier already exists and is cleaved after entering the cell, or it is formed at the outside of the membrane for the time of the passage through the membrane and cleaved again thereafter, or carrier and active agent form a unit subsisting also in the inner cell, but without negatively influencing the efficacy of the active agent.

Such properties are displayed by substances which interact either directly with the lipid bilayer of the cell membrane, interact with receptors on the cell membrane, entering the inner cell via membrane transport proteins acting as carriers or channels (ion pumps), where they change the membrane potential and thus the cellular membrane permeability. Thereby the uptake of an active agent into the cells is facilitated or accelerated, respectively. For example, oleic acid can influence the interaction with the lipids of the lipid bilayer, while cyclo-monoterpenes such as D-limones or menthol interact with the hydrocarbons of the lipid bilayer.

Primarily, the ability of substances to diffuse through a membrane into the cell corresponds directly to the substance size. Smaller molecules pass easier than larger ones. Molecules undergoing fewer hydrogen bonds also diffuse correspondingly faster than molecules eager to form hydrogen bonds.

The hydration of the polar head groups of the lipid bilayer is altered by urea or propylene glycol. Also, the removal of the hydrogen bonds leads to an accelerated uptake of the active agent (e.g. dimethyl sulfoxid, dimethylformamide, or dimethylacetamide). An increase of the moisture in the cell wall by hygroscopic substances such as pyrrolidone increases the diffusion into the cell, as well.

Also the polarity of the molecule is important. Polar and nonpolar groups can be combined here in one molecule similarly to phosphatidylcholin which is present in all cells as part of the membrane.

A further possibility is the mixture of two substances, each contributing one of the features and acting in combination as a mixture able to pass through the membrane.

Using ion pair effects resulting in a decrease of the polarity of the active agent represents a further possibility for improving the transport.

Accordingly, surfactants, for example, whose effect on the uptake of drugs decreases from anionic over cationic to non-ionic, are important and capable transport mediators.

Taking these facts into account a number of synthetic, semi-synthetic and native substances can be used to change the permeability of a cell membrane in such a way that the entering of an active agent occurs optimally.

Among such useful compounds are for example vasodilators encompassing endogenous substances as kinins, for example bradykinin, kallidin, histamine and NO synthase releasing the vasodilatory active NO from L-arginine. Substances of herbal origin such as the verifiably vasodilatory Gingko biloba extract, DMSO, xanthones, flavonoids, terpenoids, herbal and animal colorants, food dyes, NO donors as e.g. pentaerythrityl tetranitrate (PETN), carbon monoxide (CO), contrast media and contrast medium analogues belong likewise to this category.

Thus there are two possibilities which can also be combined for supporting the transport of one or several active agents into cells:
1. The transport accelerator or mediator causes an immediate substance transfer into cells limited by the exposure time with the medical device.
2. After removing the medical device the transport accelerator or mediator adheres in combination with the active agent and possibly an adhesion-supporting carrier (respectively reservoir) to the cell walls. Thus the diffusion of the active agent into the cells can occur retarded and dose-controlled.

Transport mediators, the active agent or the combination of active agents, respectively as well as a possible matrix may be applied on the medical device adhesively and/or covalently, partially or entirely covering:
1. The transport mediator and the active agent adhere adhesively and/or covalently on the medical product or on an adhesively or covalently matrix applied on the medical product.
2. The transport mediator and the active agent are covalently linked and adhere adhesively on the medical product or on a matrix adhesively or covalently applied on the medical device.
3. The transport mediator and the active agent are covalently linked and adhere covalently on the medical product or on a matrix adhesively or covalently applied on the medical product.

In many cases the effect of the mentioned substances is not limited to the transport properties, but rather they additionally display a positive effect promoting the healing. For example nitrogen monoxide produced by the cell itself is not only vasodilatory but also has antiproliferative properties. Thus all NO donors are antiproliferatives and vasodilators at the same time.

Combinations with other antiproliferative, cytotoxic and cytostatic, anti-inflammatory and also antithrombotic substances can be used herein for potentiation or complementation of the adjuvant effectiveness.

The inventive medical balloon catheters, with or without a stent, are used for prevention or reduction of restenosis particularly in in-stent-restenosis.

The temporary short-term implants are particularly suitable for the treatment and prophylaxis of vascular diseases arising from a decrease in wall shear stress or an potentially simultaneous stretch-induced increase in leucocyte adhesion and emigration. Such processes occur often at vessel bifurcations. The vessel implants according to the invention can cause an increase in wall shear stress and a strengthening or activation of smooth muscle cells (SMC), or of the vascular endothelium, respectively, thus reducing or lowering to physiological level thrombocyte adhesion and diapedesis of leucocytes present in the bloodstream. This prevents inflammatory processes and avoids, for example, chronic inflammatory bowel diseases, such as most notably Crohn's disease as well as atherosclerosis, stenosis or also restenosis.

The combination of a transmembranous transport mediator and active agent may be realized in different embodiments:
1. transport mediator and active agent are identical
2. transport mediator and active agent are not identical, but support each other in their effects.
3. the transport mediator has no influence on the effect of the added active agent and serves exclusively as a transport vehicle

On the basis of the above-mentioned different possibilities of interaction between the substances and the cell membrane, under the aspect of increasing the membrane permeability a plurality of quiet different substances exist which can function as transport mediators into the cell.

Besides the necessary biocompatibility and the molecular size of active agent and transport mediator limiting the ability of entering the cell, further physical properties of the substance are of importance. Accordingly, the substance must not be that volatile that the substance is no longer present or only in a reduced manner on the balloon, after a short period of time. That way the features of the product would be altered in such a way that a ratio of transport mediator and active action would occur which could not be defined and controlled anymore, such that the use of such a medical product would be highly questionable or made impossible, respectively. Not least a minimum storage life is absolutely necessary. Under these conditions substances with a boiling point below 100° C. cannot be used anymore, because a stable, defined product can no longer be guaranteed. Therefore, suitable substances with a boiling point above 150° C., and thus with a suitable temperature stability, are particularly preferred as transport mediators.

Consequently, the choice of the used transport mediator is directed by following basic criteria:

### 1. Biocompatibility

2. Boiling point>150° C.
3. Molecular weight (if the transport mediator is also diffusing into the cell)
4. Storage life

Further criteria have also to be considered with reference to the added active agent and limit the selection of transport mediators with regard to an optimal diffusion:
1) The necessary amount of active agent for the target which shall reach the cell during the contact time
2) Consideration of interactions with the active agent which shall be transported into the cell
3) Stability of the product
4) Guarantee of a controllable, consistent release of active agent

As mentioned, many compounds are theoretically usable as transport aid or accelerator, respectively. Depending on their mode of interaction with the membrane or the active agent they can be classified by their polarity, molecular size, their chemical group. Furthermore, combinations are possible which combine, for example, a polar and a non-polar penetration enhancer in order to obtain a significant enhancement of diffusion. Therein the combination can be made by mixing two substances or by combining the different features in one molecule.

Usually non-polar substances start interactions with the lipid bilayer. All hydrophobic molecules or molecules which are predominantly lipophilic can be used here, such as, for instance, phospholipids which have a hydrophilic head and a long hydrophobic tail of fatty acids.

Therefore phospholipids and sphingolipids, cetrimides and cetyl alcohols are well suited, because they are membrane components, so that membrane components can be seen as natural penetration enhancers, since they tend into the membrane formation.

The remarks above show that a person skilled in the art can select and use a plurality of very different compounds as transport mediators, because many substance classes can theoretically act as transport mediators; and because of this plurality of possible substances the skilled person cannot recognized which substance classes really work well and which substance groups are superior to the others, because the demands to the transport mediators are clearly more specific for balloon catheters than, for instance, for any transdermal formulations. Consequently the present invention is related to a selection of certain transport mediators and can be referred to as selection invention, and should be assessed this way also with regard to novelty and inventiveness.

According to the invention a composition is, thus, used for the coating of catheter balloons which contains at least one solvent, at least one pharmacologically active agent and a transport mediator or a mixture of transport mediators, wherein the transport mediator or the mixture of transport mediators has a boiling point of at least 150° C., the transport mediator or the mixture of transport mediators has an oily or fatty consistency at 20° C., causes no immune reaction, and the transport mediator or the mixture of transport mediators is used for coating of catheter balloons for vessel dilatation, wherein the transport mediator or the mixture of transport mediators is not a contrast agent.

It is further preferred if the transport mediator or the mixture of transport mediators and the pharmacologically active agent are contained in the composition in a molar ratio of 1:100 to 10:1, preferably of 1:50 to 2:1, and particularly preferred of 1:10 to 1:1 and, thus, in the inventive balloon coating the molar ratio of transport mediator to active agent is 1 mol:100 mol, preferably 1 mol:50 mol to 2 mol:1 mol and particularly preferred 1 mol:10 mol to 1 mol:1 mol.

The transport mediator or the mixture of transport mediators should have a boiling point of at least 150° C., preferably of at least 170° C., further preferred of at least 190° C., and yet further preferred of at least 210° C. and particularly preferred of at least 230° C.

If the physical or chemical data mentioned herein as, for instance, polarity or boiling point are related to the mixture of transport mediators, this accordingly means that the mixture itself should have these features. This can mean that all the single transport mediators being part of this mixture each have this feature, which, however, does not have to be the case necessarily.

As an example it shall be mentioned that it is preferred, among others, when the transport mediator or the mixture of transport mediators reacts pH neutrally. This statement means with regard to the mixture of transport mediators that the mixture itself reacts pH neutrally. This is realized, if for example all ingredients of the mixtures, i.e. all single transport mediators themselves are pH neutral, or if the pH influences of the single transport mediators are neutralizing each other, so that the single transport mediators as parts of the mixtures are not pH neutral, but their mixture all together is pH neutral.

It is further preferred if the transport mediator is not a polymer and the mixture of transport mediators does not contain a polymer as transport mediator.

Further preferred are transport mediators which have at least 6 carbon atoms or at least two oxygen atoms or at least one nitrogen atom, as well as mixtures of such transport mediators.

It is also preferred, if the transport mediator or the mixture of transport mediators has a vapor pressure of less than 100 Pascal, preferably of less than 65 Pa, more preferred of less than 25 Pa and particularly preferred of less than 6 Pascal at 20° C. For comparison there are some vapor pressures shown in the following:
Ethyl acetate 9700 Pa at 20° C.
DMSO 250 Pa at 20° C.
Camphor 20 Pa at 20° C.
Ethylene glycol 5,3Pa at 20° C.

Preferred are transport mediators which are lipophilic and have a partition coefficient between butanol and water of ≧0.5.

Further preferred are transport mediators which have at least one ionic or ionizable functional group.

Preferred is further a transport mediator, which is able to pass through the plasma membrane.

Preferred is also a transport mediator with a molecular weight of 100 g/mol to 1000 g/mol, further preferred of 200 g/mol to 900 g/mol, yet further preferred of 300 g/mol to 800 g/mol, yet further preferred of 400 g/mol to 700 g/mol and particularly preferred of 500 g/mol to 600 g/mol.

As preferred can also transport mediators be referred to which are lipophilic and hydrophilically esterified in such a way that the transport mediators have a partition coefficient between butanol and water of ≦0.5.

Preferred is also a transport mediator or a mixture of transport mediators which has a pH value of 5<pH<7 in aqueous solution.

Preferred is also a transport mediator or a mixture of transport mediators which is able to increase the moisture of the cell wall.

It is also important that such transport mediators or such mixtures of transport mediators are not preferred which form micelles, and particularly micelles which are hydrophilic to the outside.

As preferred can a transport mediator referred to which is able to form hydrogen bonds.

Preferred are also transport mediators and mixtures of transport mediators which can interact with the lipids of the lipid bilayer and/or with the hydrocarbons of the lipid bilayer.

Preferred are also transport mediators which are hydrophilic and esterified hydrophobically in such a way that the transport mediators have a partition coefficient between butanol and water of ≧0.5.

Further preferred are transport mediators or mixtures of transport mediators which have a pH value of 9>pH>7 in aqueous solution.

Preferred are also transport mediators or mixtures of transport mediators which are able to alter the membrane potential in such a way that the uptake of active agent of the cell is accelerated or the membrane potential gradient leads to an accelerated uptake of the active agent into the cell, respectively.

Preferred are also transport mediators or mixtures of transport mediators which are able to alter the diffusion potential in a way that the uptake of the active agent of the cell or the diffusion into the cell is accelerated, respectively.

Preferred is also a transport mediator which is able to cleave the hydrogen bonds in the cell wall.

Preferred are also transport mediators or mixtures of transport mediators which will be volatized at most 50% by weight, preferably at most up to 40% by weight, further preferred at most 30% by weight, further preferred at most 20% by weight, and particularly at most 15% by weight, at 25° C. after 2 months.

Preferred is also a transport mediator which alters the thermodynamic condition of the added active agent in such a way that the diffusion into the cells is enhanced.

Particularly preferred are transport mediator subgroups which are outstanding by a particular suitability for the inventive use in comparison to the large group of possible transport mediators. This are primarily: amides, phenols, phenolic esters, phenolic ethers, aromatic alcohols, aromatic acids, sulfoxides, organic boron compounds, polyvalent alcohols with 2 to 6 carbon atoms, monoglycerides of fatty acids and alcohols, fatty acid ethers, terpene hydrocarbons, alcohols with at least 8 carbon atoms, heterocyclic compounds, alkaloids, nanoparticles, enzymes and quaternary ammonium salts.

As practical examples following explicit representatives can be named for the following transport mediator subgroups:
Amides and amines such as urea, DMF, DMA, cyclo phosphamide, alkanolamide such as 1,2,3-propantriol homopolymer (Z)-9-octadecenoate, decyloxy- and octyloxy-poly-oxyethylen-polyoxypropylene, 2-hydroxyethyl ethylene diaminetriacetic acid, 1,5-pentamethylene glycol, aspartame;
Phenols, phenolic esters, phenolic ethers such as anisole, t-anethole, thymol, carvacrol, chlorocresol, octyl phenol ethoxylate; particularly vanillin, coniferyl alcohol and coniferin;
Aromatic alcohols and aromatic acids such as salicylic acid, salicylic alcohol, phenylethanol, cinnamyl alcohol, adrenaline, dopamine, amphetamine; particularly ferulic acid, curcumin and caffeic acid;
Sulfoxides such as all biocompatible sulfoxides, diethyl sulfoxides and acesulfam K;
Organic boron compounds: all biocompatible boric acid ester, phenylborates and metaborates; particularly phenylboronic acid;
Polyvalent alcohols with 2 to 6 carbon atoms such as lactitol, mannitol, dulcitol, isomalt, sucrose, xylitol, 2-ethyl-1,3-hexanediol, particularly alitame, maltitol
Monoglycerides of fatty acids and alcohols such as glycerine monooleate, glycerine monolinoleate, glycerine monolaurate, maltol, meglumine, and commonly acyl glycerides
Glycol ether, ethylene glycol monoether, ethylene glycol diether, propylene glycol monoether, particularly propylene glycol diether;
Fatty acid ethers and carboxylic acid ethers with at least 8 carbon atoms such as polyoxyethylene lauryl ether, polyethylene glycol monolauryl carboxymethyl ether, particularly diethylene glycol lauryl ether;
High-boiling hydrocarbons with at least 8 carbon atoms and their mixes such as terpene hydrocarbons:

Monocyclic Terpenes:
menthol, limonene, isoprene, α, β and gamma-terpineols, 1,8-terpin, ascaridole, carvone, pulegone, particularly thymol, 1,8-cineol;

Bicyclic Terpenes (Caran, Pinan, and Bornan Group):
α-pinen, 3-carene, camphene, particularly borneol and camphor;

Monocyclic Sesquiterpenes:
bisabolene and farnesol;

Acyclic terpenes: myrcene, phellandrene and ocimene, linalool;

Tricyclic Sesquiterpenes:
santalene;

Triterpenes (Squalenoids):
squalen; tetracyclic triterpenic acid;
Tetraterpenes: carotinoids such as carotene, lycopine, zeaxanthin, lutein and lutein in combination with zinc oxide, crocetin, commonly lipochromes;

Polyprenes:
male and female steroid hormones (androgens, estrogens, and gestagens): testosterone, androsterone, estriol, estradiol, estrone, clomifene (INN), prolutone (INN: progesterone), synthetic estrogens, particularly fosfestrol and tibolone;

Corticoids:
cortisol (INN, cortisone), aldosterone (INN), triamcinolone (INN);
Alcohols with at least 8 carbon atoms such as alcanols, myristyl alcohol, stearyl alcohol, sterols, alkyl-2-(N,N-disubstituted amino)-alkanoates and alkyl-2-(N,N-disubstituted amino)-alkanol alkanoates, 1,2,3-butanetriol, 1,2,4-butanetriol, 1,2-butanediol, 1,3-butanediol, 1,4-butanediol, 1,2,3,4-butanetetraol, glycerol, glycol, wool fat alcohols such as lanolin;
Heterocyclic compounds such as N-methylpyrrolidone, bilirubin, biotin, sulfamethoxazole (INN), particularly ascorbic acid ether and hydrophobically esterified ascorbic acids, such as ascorbyl palmitate;
Alkaloids and derivates such as 1-substituted azacylcloalkan-2-one, laurocapram (=1-dodecylazacycloheptan-2-one) and derivatives, cyclodextrins, azacyclo alkenes, chlorhydrin;
Nanoparticles such as fullerene-based peptides;
Enzymes such as hyalourodinase, streptodornase, chymotrypsin, bromelain, papain, deoxyribonuclease, collagenase, serine proteases;
Quaternary ammonium salts such as 2,3-epoxypropyltrimethylammonium chloride (QUAB 151), 3-chloro-2-hydroxypropyltrimethylammonium chloride (QUAB 181), dodecyl-, hexadecyl-, tetradecyltrimethylammonium halide, glycidyltrimethylammonium halide, 3-chloro-2-hydroxypropyl trimethylammonium halide, benzethonium halide, wherein halides refer to: fluoride, chloride, bromide, iodide;
Fumarates such as sodium stearyl fumarate, fumaric acid, fumaric acid ether;
Phosphates such as alkyl-(polyoxyethyl)-phosphate;
Polysaccharides such as carrageenan, sorbitol, sorbitol ether sucrose, hydrophobically esterified respectively etherified xylitol respectively glucose, maltatol, mannitol, meglumine.

Particularly preferred are also tartrates as well as tartaric acid esters of the following formula:

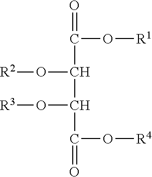

wherein
R¹, R², R³ and R⁴ represent independently from each other hydrogen, —CH₃, —C₂H₅, —C₃H₇, CH(CH₃)₂, C(CH₃)₃, C₄H₉, —CH₂—CH(CH₃)₂, —CH(CH₃)—C₂H₅, —C₅H₁₁, —C₆H₁₃, —C₇H₁₅, —C₈H₁₇, -cyclo-C₃H₅, -cyclo-C₄H₇, -cyclo-C₅H₉, -cyclo-C₆H₁₁, or an alkyl, arylalkyl or cycloalkyl residue which are linear or branched, saturated or unsaturated, substituted or unsubstituted with at least one functional group.
As functional groups for substitution at the alkyl, arylalkyl or cycloalkyl residue the following moieties are possible:
—H, —OH, —OCH₃, —OC₂H₅, —OC₃H₇, —O-cyclo-C₃H₅, —OCH(CH₃)₂, —OC(CH₃)₃, —OC₄H₉, —SH, —SCH₃, —SC₂H₅, —NO₂, —F, —Cl, —Br, —I, —COCH₃, —COC₂H₅, —COC₃H₇, —CO-cyclo-C₃H₅, —COCH(CH₃)₂, —COOH, —COOCH₃, —COOC₂H₅, —COOC₃H₇, —COO-cyclo-C₃H₅, —COOCH(CH₃)₂, —OOC—CH₃, —OOC—C₂H₅, —OOCC—C₃H₇, —OOC-cyclo-C₃H₅, —OOC—CH(CH₃)₂, —CONH₂, —CONHCH₃, —CONHC₂H₅, —CONHC₃H₇, —CONH-cyclo-C₃H₅, —CONH[CH(CH₃)₂], —CON(CH₃)₂, —CON(C₂H₅)₂, —CON(C₃H₇)₂, —CON(cyclo-C₃H₅)₂, —CON[CH(CH₃)₂]₂, —NHCOCH₃, —NHCOC₂H₅, —NHCOC₃H₇, —NHCO-cyclo-C₃H₅, —NHCO—CH(CH₃)₂, —NHCO—OCH₃, —NHCO—OC₂H₅, —NHCO—OC₃H₇, —NHCO—O-cyclo-C₃H₅, —NHCO—OCH(CH₃)₂, —NHCO—OC(CH₃)₃, —NH₂, —NHCH₃, —NHC₂H₅, —NHC₃H₇, —NH-cyclo-C₃H₅, —NHCH(CH₃)₂, —NHC(CH₃)₃, —N(CH₃)₂, —N(C₂H₅)₂, —N(C₃H₇)₂, —N(cyclo-C₃H₅)₂, —N[CH(CH₃)₂]₂, —SO₂CH₃, —SO₂C₂H₅, —SO₃₁₄, —SO₃CH₃, —SO₃C₂H₅, —OCF₃, —OC₂F₅, —NH—CO—NH₂, —NH—C(═NH)—NH₂, —O—CO—NH₂, —O—CO—NHCH₃, —O—CO—N(CH₃)₂, —O—CO—N(C₂H₅)₂, —CH₂F, —CHF₂, —CF₃, —CH₂Cl, —CH₂Br, —CH₂—CH₂F, —CH₂—CF₃, —CH₂—CH₂Cl, —CH₂—CH₂Br, —CH₃, —C₂H₅, —C₃H₇, —CH(CH₃)₂, —C(CH₃)₃, —C₄H₉, —CH₂—CH(CH₃)₂, —CH(CH₃)—C₂H₅, —OS₅H₁₁, —C₆H₁₃, —C₇H₁₅, —C₈H₁₇, -cyclo-C₃H₅, -cyclo-C₄H₇, -cyclo-O₅H₉, -cyclo-C₆H₁₁, -Ph, —CH₂-Ph, —CH═CH₂, —CH₂—CH═CH₂, —C(CH₃)═CH₂, —CH═CH—CH₃, —C₂H₄—CH═CH₂, —CH═C(CH₃)₂, —CH₂—C≡CH.

Preferred are the aforementioned alkyl groups, substituted alkyl groups as well as diesters, triesters and particularly tetraesters of tartaric acid. Preferred tartrates are tetramethyl tartrate, tetraethyl tartrate, tetrapropyl tartrate, tetrabutyl tartrate.

Preferred is also a transport mediator with a molecular weight of 150 g/mol to 390 g/mol, further preferred of 170 g/mol to 370 g/mol, yet further preferred of 190 g/mol to 350 g/mol, yet further preferred of 200 g/mol to 330 g/mol, and particularly preferred of 230 g/mol to 310 g/mol.

### Base Coating

A polymer coating or a base coating can be present on the catheter balloon below the coating of the present invention made of transport mediator and active agent.

Biocompatible substances can be used for the base coating which, as a minimum demand, do not negatively influence the properties and the use of the implants as compared to uncoated implants.

The following biocompatible biodegradable or/and biostable polymers are preferably used for the coating of the catheter balloons:

As biologically stable or only slowly degradable polymers can be named: polyacrylic acid and polyacrylates such as polymethylmethacrylate, polybutylmethacrylate, polyacrylamide, polyacrylonitriles, polyamides, polyetheramides, polyethylenamine, polyimides, polycarbonates, polycarbourethanes, polyvinylketones, polyvinylhalogenides, polyvinylidenhalogenides, polyvinylethers, polyvinylaromates, polyvinylesters, polyvinylpyrollidones, polyoxymethylenes, polyethylene, polypropylene, polytetrafluoroethylene, polyurethanes, polyolefine elastomers, polyisobutylenes, EPDM gums, fluorosilicones, carboxymethylchitosane, polyethylenterephthalate, polyvalerates, carboxymethylcellulose, cellulose, rayon, rayon triacetates, cellulose nitrates, cellulose acetates, hydroxyethylcellulose, cellulose butyrates, cellulose acetate-butyrates, ethylvinyl acetate copolymers, polysulfones, polyethersulfones, epoxy resins, ABS resins, EPDM gums, silicon prepolymers, silicones such as polysiloxanes, polyvinylhalogenes and copolymers, cellulose ethers, cellulose triacetates, chitosane, chitosane derivatives, polymerizable oils such as linseed oil and copolymers and/or mixtures thereof.

As biologically degradable or resorbable polymers can be used e.g.: polyvalerolactones, poly-ε-decalactones, polylactides, polyglycolides, copolymers of the polylactides and polyglycolides, poly-ε-caprolactone, polyhydroxybutyric acid, polyhydroxybutyrates, polyhydroxyvalerates, polyhydroxybutyrate-co-valerates, poly(1,4-dioxane-2,3-diones), poly(1,3-dioxane-2-one), poly-para-dioxanones, polyanhydrides such as polymaleic anhydrides, polyhydroxymethacrylates, fibrin, polycyanoacrylates, polycaprolactonedimethylacrylates, poly-β-maleic acid, polycaprolactonebutyl-acrylates, multiblock polymers such as from oligocaprolactonedioles and oligodioxanonedioles, polyetherester multiblock polymers such as PEG and polybutyleneterephtalate, polypivotolactones, polyglycolic acid trimethyl-carbonates, polycaprolactone-glycolides, poly(g-ethylglutamate), poly(DTH-iminocarbonate), poly(DTE-co-DT-carbonate), poly(bisphenol-A-iminocarbonate), polyorthoesters, polyglycolic acid trimethyl-carbonates, polytrimethylcarbonates, polyiminocarbonates, poly(N-vinyl)-pyrrolidone, polyvinyl alcohols, polyesteramides, glycolated polyesters, polyphosphoesters, polyphosphazenes, poly[p-carboxyphenoxy)propane], polyhydroxypentanoic acid, polyanhydrides, polyethyleneoxide-propyleneoxide, soft polyurethanes, polyurethanes having amino acid residues in the backbone, polyether esters such as polyethyleneoxide, polyalkeneoxalates, polyorthoesters as well as their copolymers, carrageenanes, fibrinogen, starch, collagen, protein based polymers, polyamino acids, synthetic polyamino acids, zein, modified zein, polyhydroxyalkanoates, pectic acid, actinic acid, modified and non modified fibrin and casein, carboxymethylsulfate, albumin, furthermore hyaluronic acid, heparan sulfates, heparin, chondroitine sulfate, dextran, β-cyclodextrines, copolymers with PEG and polypropylene glycol, gum arabic, guar, gelatine, collagen, collagen-N-hydroxysuccinimide, modifications and copolymers and/or mixtures of the aforementioned substances.

Particularly preferred polymers are polysulfones, polyethersulfones, silicones, chitosan, polyacrylates, polyamides, polyetheramides, polyurethanes, polylactides, polyglycolides, copolymers of polylactides and polyglycolides, polyhydroxybutyric acid, polyhydroxybutyrates, polyhydroxyvalerates, polyhydroxybutyrate-co-valerates, poly(1,4-dioxane-2,3-diones), poly(1,3-dioxane-2-one), poly-para-dioxanones, polyanhydrides, polyester, PEG, hyaluronic acid, heparan sulfate, heparin, chondroitin sulfate, dextram and β-cyclodextrins.

Balloon with a Crimped Stent

A further preferred embodiment of the present invention comprises an inventive catheter balloon with a crimped stent.

In this embodiment there are particularly 4 variants to be selected and used depending on the vessel stenosis that needs treatment.

Variant [A] represents a catheter balloon with a crimped non-resorbable and uncoated stent.

In variant [B] the non-resorbable stent is coated with a substance-releasing carrier system.

Variant [C] comprises a resorbable uncoated stent and variant [D] is a catheter balloon with a resorbable substance-releasing stent.\

Variant [A]: Since a substance-releasing system, generally a substance-releasing coating, on the stent is not always desirable and in some cases the problem of late thrombosis may occur variant [A] offers an ideal system for keeping open a severely constricted corporal lumina as for example the bile duct, oesophagus, unitary tract, pancreas, renal tract, pulmonary tract, trachea, small intestine and large intestine and particularly blood vessels with a permanent stent without a coating, wherein however the application of an active agent is not prohibited.

The catheter balloon according to variant [A] is coated with a pure layer of active agent or a carrier containing an active agent, and during dilation on the one hand the stent is placed and on the other hand an active agent is applied at least over the whole length of the stent, and advantageously beyond, which enables a controlled incorporation and prevents an overgrowing of the stent with mostly smooth muscle cells. As an active agent or mixture of active agents the abovementioned active agents and especially paclitaxel and/or rapamycin can be applied.

Preferably the catheter balloon is coated with an active agent with or without a carrier system in such a way that the balloon coating extends both stent ends, preferably by 10-20% of total stent length over an end of the stent. Thus the active agent is transferred during dilation also to the section of the vessel at both ends of the stent, to where the stent does not reach, and the active agent is transferred all over the vascular wall located between the expanding or expanded stent struts, respectively.

This embodiment has the advantage that the stent surface does not have an active agent inhibiting or killing cells, particularly smooth muscle cells, which are in direct contact with the stent surface. In contrast, a sufficient amount of the active agent is applied in the recesses between the stent struts so that consequently the rapid overgrowth of the stent starting from the recesses and continuing to the inside of the stent which eventually leads to in-stent restenosis is contained or reduced to a tolerable degree, respectively.

As a substance-coated stent releases the active agent only from its surface and not from the recesses of the stent struts or from the end of the stent or respectively the area extending it and moreover releases it consequently to the adjacent tissue which should not be inhibited or killed, according to variant [A] the active agent is precisely applied where it is needed, almost covering the entire area. It is further preferred when the catheter balloon is coated at its distal and proximal end for some mm beyond the end of the stent so that the covering of the vascular wall with the active agent extends the end of the stent by some mm for providing also the terminal sections of the stent sitting in the vessel with a sufficient amount of the active agent.

It is further preferred when the catheter balloon is coated at its distal and proximal end for some mm beyond the end of the stent so that the covering of the vascular wall with the active agent extends the end of the stent by some mm for providing also the terminal sections of the stent sitting in the vessel with a sufficient amount of the active agent.

Thus preferably the catheter balloon is coated according to the present invention and subsequently an uncoated stent is crimped onto the balloon.

Variant [B] can be achieved when a non-resorbable stent as in variant [A] is crimped onto a balloon and subsequently the stent and the balloon are coated with an active agent according to the present invention.

The term “non-resorbable” means that the stent is a permanent implant which will not or only very slowly be dissolved under physiological conditions. Such stents are made of, for example, medical high quality steel, titanium, chromium, vanadium, tungsten, molybdenum, gold, nitinol, magnesium, zinc, iron, alloys of the aforementioned metals as well as ceramics or also biostable polymers.

When coating simultaneously a catheter balloon with a crimped stent, then preferably a solution of the active agent and the at least one transport mediator is used in a solvent which affects the catheter balloon as little as possible but nevertheless preferably is wetting and additionally sufficiently fluid to flow between the struts of the crimped stent when being compressed.

This embodiment is suitable for a spontaneous release of a relatively huge amount of active agent, since the recesses of the stent struts and the recesses between the inner surface of the stent and the surface of the catheter balloon serve as a reservoir for the active agent.

The difference to variant [A] consists mainly in the applicable amount of the active agent, as according to the above described method a considerably larger amount of an active agent or mixtures of active agents can be applied on the stent and the catheter balloon.

For hydrophobic active agents such as paclitaxel solutions made of e.g. dimethyl sulfoxide (DMSO), chloroform, ethanol, acetone, methyl acetate and hexane and their mixtures or for e.g. rapamycin solutions made of acetic acid ethyl ester, methanol/ethanol mixtures, ethanol/water mixtures or ethanol are suitable for a coating solution. Of course also other active agents in suitable solvents or solvent mixtures can be used.

It is also possible to add an additive to the solution with the active agent wherein polymeric additives, however, are rather seldom used when the catheter balloon is coated together with the crimped stent. If a carrier system shall be used rather non-polymeric carriers as for example contrast media or contrast media analogues as well as biocompatible organic substances are suitable which improve the coating properties and enhance the uptake of the active agent into the vessel, as for example amino acids, sugars, vitamins, saccharides, 2-pyrrolidone, acetyltributyl and acetyltriethyl citrate, tributyl and triethyl citrate, benzoic acid benzyl ester, triethyl and dimethyl phthalate, fatty acid esters such as isopropyl myristate and palmitate, fatty acid ether and the like. Mixtures of these different substances turned out to be also well suitable. Accordingly, the mixture of the polysaccharides carrageenan, lecithin and glycerine, for example, proves to be an extremely suitable carrier system. Also, physiologically acceptable salts can be used as a matrix for embedding the active agent.

Also in this variant the balloon is preferably coated beyond the surface covered by the stent. The coated section of the balloon extending beyond the stent ends does preferably not exceed 20% of total stent length, more preferred not more than 15% and particularly preferred not more than 10% of total stent length.

Generally a whole coverage coating is advantageous in variant [A] as well as in variant [B], i.e. the catheter balloon according to variant [A] or the stent and the catheter balloon according to variant [B] are provided with a coating covering the entire area.

The variants [A] and [B] can additionally be modified in such a way that the coating with the active agent does not happen uniformly, but upon using a gradient, i.e. a concentration gradient of the active agent on the balloon, or balloon and stent surface is generated. Accordingly, a higher concentration of active agent can, for example, be applied on the middle of the balloon, or at one or both ends of the catheter balloon or on the middle and at one or both ends.

Furthermore, only at one position or section of the catheter balloon a higher concentration of the active agent can be applied than on the rest of the surface. Accordingly, the ends of the stent, for example, require special attention particularly in the early phase after the implantation since these transitional sections have a higher risk. Here, any variation is conceivable.

Further, in a step prior to the coating step, a hemocompatible layer can be applied adhesively or preferably covalently to the uncoated surface of the medical product or may be immobilized by cross-linking, e.g. with glutardialdehyde on the surface of the medical product. A layer which does not activate the blood coagulation in such a way makes sense because thereby the hemocompatibility of the surface of the medical product is enhanced and the risk of thrombosis is reduced. This coating step is particularly reasonable most of all when the short time implant shall be coated only partially. The section not coated with active agent has thus advantageously a coagulation non-activating, athrombogenic surface and thus guarantees a considerably higher safety during and after the contact of the medical product with the blood.

Additionally a preferably hemocompatible layer may be applied on the stent as a permanent implant, which is prepared of the following preferred compounds: heparin of native origin as well as of regioselectively produced derivatives with different degrees of sulfation and acetylation in the molecular weight range of pentasaccharide being responsible for the antithrombotic effect up to the standard molecular weight of commercially available heparin of about 13 kD, heparan sulfates and their derivatives, oligo- and polysaccharides of the erythrocyte glycocalix, oligosaccharides, polysaccharides, completely desulfated and N-reacetylated heparin, desulfated and N-reacetylated heparin, N-carboxymethylated and/or partially N-acetylated chitosan, polyacrylic acid, polyether ether ketone, polyvinylpyrrolidone, and/or polyethylene glycol as well as mixtures of these substances.

### Coating Methods

A further aspect of the present invention is directed to methods for coating balloon catheters, comprising the following steps:
a) providing a composition containing at least one solvent, at least one pharmacological agent and a transport mediator or a mixture of transport mediators, wherein the transport mediator or the mixture of transport mediators has a boiling point of at least 150° C., the transport mediator or the mixture of transport mediators has an oily or solid consistency at 20° C., causes no immune reaction, and the transport mediator or the mixture of transport mediators is used for the coating of a catheter balloon for dilation of vessels, wherein the transport mediator or the mixture of transport mediators is not a contrast agent;
b) providing a balloon catheter with catheter balloon;
c) coating of the catheter balloon by squirting method, pipetting method, capillary method, fold spray method, drag method, thread drag method or rolling method;
d) drying of the coating on the balloon surface or removal of the solvent.

Further the present invention is related to balloon catheters which can be obtained by one of the methods disclosed herein. The coating methods such as e.g. squirting method, pipetting method, capillary method, fold spray method, drag method, thread drag method or rolling method are described in detail in the following. The catheter balloons can be coated with or without a stent.

The inventive balloon catheters contain, have or comprise a catheter balloon with a dried oily or solid coating of at least one pharmacological active agent and a transport mediator or a mixture of transport mediators, wherein the transport mediator or the mixture of transport mediators has a boiling point of at least 150° C., the transport mediator or the mixture of transport mediators has an oily or solid consistency at 20° C., causes no immune reaction, and the transport mediator or the mixture of transport mediators is used for the coating of a catheter balloon for dilation of vessels, wherein the transport mediator or the mixture of transport mediators is not a contrast agent. Balloon catheters coated that way are outstandingly well suited for the dilation and opening of corporal lumina, especially of blood vessels and particularly in the cardiovascular field and thus for the prophylaxis and treatment of stenoses and restenoses.

The selection of transport mediators is a significant part of the invention and the selectively chosen transport mediators for the herein described purpose were described in the chapter “transport mediators” in detail.

The catheter balloon is either completely or partially coated with a solution of the substances to be applied including the active agent or the combination of active agents by a spraying, dipping, brushing, squirting, drag, roll, pipetting method or electro-spinning, or completely or partially coated with a matrix.

As solvents volatile organic compounds such as e.g. dichloromethane, chloroform, ethanol, acetone, heptane, n-hexane, DMF, DMSO, methanol, propanol, tetrahydrofuran (THF), methylenechloride, ether, benzine, acetonitrile, acetic acid ethyl and methyl ester, cyclohexane and corresponding mixtures thereof are used. Depending on the coating material (e.g. hydrogels or water-soluble active agents) also the presence of water may be desirable.

When choosing the solvent it is in general most of all important that it does not dissolve the material of the catheter balloon or render it useless, or the exposure time of the solvent is that short that no damages can occur.

The catheter balloon either in the expanded or in the folded state can be coated, partially or completely coated, coated selectively under the folds or coated together with a mounted (crimped) stent.

The coating can be done by a spraying, dipping, brushing, squirting, dragging, rolling, and/or pipetting method. The pipetting, dragging, rolling or squirting methods are particularly suitable for the use on folded catheter balloons or fold balloons as with these methods the solution with the active agent or with the combination of active agents to be applied can be specifically applied into or under the folds. It is important thereby that no impairment in functionality comes along with this partial coating. Accordingly, the folds may, for example, not stick together when being expanded and thus counteract the expansion Likewise the nominal pressure on the balloon should not be forced to be increased beyond the maximum value in order of being able to counteract adhesive forces of the coating in the folds. Uneven expansion should also be avoided. The coating shall in no case impair the expansion characteristics of the balloon catheter.

Further, the catheter balloon can be coated together with a crimped stent, or a bare stent as well as an already coated stent can be crimped onto the coated catheter balloon such that a system can be achieved of for example an active agent rapidly released from the catheter balloon and an active agent slowly released from the coating of the stent.

In combination with a stent coated on his part and able to release an active agent a substance-releasing balloon catheter is particularly advantageous in the early phase of the healing process, as only that way the full-coverage contact with the sector to be treated can be realized and the active agent enters the affected vascular wall throughout the entire area. The whole affected sector is provided with active agent when being exposed to the surface of the balloon catheter while the stent with a preferably small surface area covers only a small portion of the surface of the vascular wall.

The advantage should be realized in the same manner for the outer sections of the stent which continuously cause problems. A catheter balloon capable of releasing active agent also in the outer sections delivers an optimal supply for the vessel even into the problem areas of the stent.

The catheter balloons with a specially made surface are preferably coated with the spraying or pipetting method. In the spraying method the catheter balloon is suspended in a rotating manner and the shape of the catheter balloon is stabilized by a light vacuum. For example, by this it can be prevented that the folds of a fold balloon during rotating flip or skid and thus the coating cannot be specifically locally performed.

The balloon catheter tethered in such a way is briefly sprayed several times while drying intermittently. If desired, an outer protective layer or barrier layer is also preferably applied by the spraying method. The same applies for pure active agent—transport mediator—layers containing for example paclitaxel or rapamycin which are also applied preferably by the spraying method.

The pipetting method is particularly suitable for the coating of a balloon catheter. Herein the rotatably tethered balloon catheter (with or without a stent) is coated by means of a fine nozzle prolonged with a capillary and through which the coating solution exits longitudinally onto the balloon catheter.

In the squirting or pipetting method a fine nozzle or cannula is moved under the folds for the preferable filling of the folds of a fold balloon, and the solution to be applied is squirted into the fold wherein preferably the nozzle or cannula is moved along the fold or, when the nozzle or cannula is stationary the fold balloon is moved longitudinally to the fold. This method allows for a very precise and exact coating of each single fold or of the whole balloon, respectively. A possibly used solvent evaporates or is removed under vacuum.

If the consistency of the mixture or solution to be applied allows flowing into the folds, the fold balloon is then positioned horizontally with one fold upside, or preferentially inclined by 5 to 25 degrees, so that the syringe or nozzle can be set at the lower end of the fold balloon at the orifice of the fold, and the mixture can flow on its own into the fold and fill it up completely. As soon as the mixture has a consistency at which it can no longer flow out of the fold the fold balloon is turned and the next fold is filled until generally all folds of the balloon are filled. Fold balloons are coated preferably in the compressed state, whereby some special embodiments of fold balloons can be coated also in the expanded state.

Such a coating method comprises the steps
a) providing a fold balloon,
b) placing a fold of the balloon into a horizontal position or inclined up to 25 degrees,
c) setting the orifice of the syringe at the orifice of the fold which faces the top of the balloon,
d) performing a relative movement of the orifice of the syringe and the fold balloon longitudinal to the fold
e) filling the fold during the movement with a mixture of an active agent and a transport mediator in a suitable solvent,
f) if necessary, drying of the mixture inside the fold to such a degree that leaking of the mixture out of the fold is prevented,
g) rotating the balloon by 360° divided by the number of folds
h) repeating the steps b) to g) until all folds are filled, and
i) drying of the mixtures inside the folds until the mixture solidifies.

If mixtures of lower viscosity are used, the orifice of the syringe is set in step c) at the bottom end and the fold is filled without a relative movement according to step d) mainly because of capillary forces.

The present invention is further directed to a method of keeping open stenotic vessel lumina, especially of cardiovascular vessels by means of short-term dilation. In this method a catheter balloon without a stent is expanded within at most 50 seconds, preferably at most 40 seconds, more preferably at most 30 seconds and most preferably at most 20 seconds and then re-compressed to a diameter less than the 1.5 fold initial diameter in the compressed state, wherein during this procedure the vessel is only overstretched up to at most 10% of its diameter in the non-stenotic state and at least 20% of the contained active agent per mm² surface of the balloon is released and mostly transferred onto the vascular wall.

Herein the transfer of the active agent does preferably not occur in its pure form but in a matrix of transport mediators which is active as a reservoir for the active agent for at least one hour after dilation and which releases further active agent to the vascular wall before being dissolved or degraded.

This method thus is characterized in transferring a preferably large amount of active agent locally and specifically onto the vascular wall of a stenotic section of a vessel during a preferably short time and in providing a local reservoir of active agent during the following 30 to 60 minutes up to maximally 3 days, which is dissolved or degraded thereafter.

In this method especially active agents combining anti-inflammatory and antiproliferative properties turned out to be particularly suitable (see list of active agents p. 8-10). Among them are for example colchicine, angiopeptin, but above all rapamycin and its derivatives, furthermore other hydrophobic active agents, particularly paclitaxel and paclitaxel derivatives have been shown to be very suitable.

The fold coating methods or fold filling methods according to the present invention are the pipetting method, also named capillary method, the squirting method and the spray method, also named fold spray method, in order to clarify the difference to the unselective spray method for the entire catheter balloon.

Thus the present invention relates to methods for coating or filling the folds of a catheter fold balloon in the following manner:
a) a composition containing an active agent is released at the distal or the proximal end of a fold of the catheter fold balloon and the fold is filled by capillary forces; or
b) a syringe releasing a continuous flow of a composition containing an active agent is moved along the fold relatively to the catheter fold balloon; or
c) a plurality of aligned release orifices is moved under the folds of the fold balloon and a composition containing an active agent is released simultaneously from the plurality of release orifices into the fold.

It is of advantage that this coating or filling method can be carried out preferably in the compressed or deflated or at most 10% inflated state of the catheter balloon. The term “10% inflated state” means that the catheter balloon has undergone 10% of inflation, i.e. expansion of the maximum expansion planned during dilation. If the expansion planned during dilation is referred to as 100% and the deflated state is set to 0% 10% of inflation results from the following formula:

$$( diameter of the deflated catheter balloon ) + ( diameter of the inflated catheter balloon - diameter of the deflated catheter balloon ) / 10$$

Further, several or all folds can be coated or filled simultaneously according to the methods of the invention, and the coating or filling can be performed specifically. A specific filling of the folds or coating of the folds means that only the folds are filled or coated and the surface of the catheter balloon outside the folds will not be coated.

A preferably used composition of active agent, solvent and matrix such as contrast medium has the consistency of a paste, gel, viscous mass or a viscous dispersion or emulsion or a tough pap.

This composition has the advantage that it maintains its consistency during the coating. This paste or (highly) viscous mass or thick suspension is applied into the folds under pressure with a squirting device, preferably a nozzle.

If necessary, the nozzle can widen the folds of the balloon and specifically fill the cavities formed by the folds. Fold balloons usually have 4 or more folds which will be filled individually.

It turned out to be particularly advantageous to rotate the fold balloon in the direction of the orifices of the folds after filling of one or several or all folds. This rotation leads to a complete and even distribution of the viscous paste in the folds and to a release of possible air locks. After rotating of the fold balloon a further filling of already filled or empty folds can be done.

During and/or after the rotation the composition in the folds dries either under atmospheric or diminished pressure. Drying or hardening of the composition occurs by removing the at least one solvent by evaporation. The dried composition has a porous consistency and can very easily be detached from the balloon surface during dilation. The solvent has been removed except for the usual residuals and the contrast medium forms a porous matrix for the active agent and is additionally capable to release the active agent rapidly and in high concentration after dilating the fold balloon. Moreover, the inventive method has the advantage to work very material-sparing since only the folds are coated or filled and thus no active agent is located on the outer surface of the balloon which could get lost during the introduction of the catheter.

### General Description of the Coating Methods

### Pipetting Method—Capillary Method

This method comprises the following steps:
a) providing a folded compressed catheter balloon,
b) providing a coating device with an outlet capable for point-shaped release of the coating solution,
c) setting the outlet capable for point-shaped release of the coating solution at the proximal or at the distal end of a fold of the catheter balloon,
d) releasing a defined amount of the coating solution through the outlet at the proximal or distal end of a fold, and
e) filling the fold with the coating solution because of capillary effects.
   Optionally, step f) for drying can still follow:
f) drying of the coating solution in the fold wherein the catheter balloon is rotated during drying about its longitudinal axis in direction of the orifice of the folds.

This method coats or fills specifically the folds and can be performed with any coating solution which is still viscous in such a way that it is drawn into the fold during 5 minutes because of capillary forces or by additionally using gravitation, preferably 2 minutes, and the fold is more or less completely filled.

Under the term coating solution as used herein it is understood the composition used according to the present invention containing at least one solvent, at least one pharmacologically active agent and a transport mediator or a mixture of transport mediators, wherein preferably the substance classes of transport mediators described herein can be used as transport mediators.

### Squirting Method or Syringe Method:

This method comprises the following steps:
a) providing a folded compressed catheter balloon,
b) providing a coating device with at least one nozzle or at least one syringe-shaped outlet,
c) setting the nozzle or the outlet at the proximal or at the distal end of a fold of the catheter balloon,
d) moving the nozzle or the outlet along the fold relatively to the fold, and
e) releasing a defined flow of coating solution per time and per covered distance.
   Optionally, step f) for drying can still follow:
f) drying of the coating solution in the fold or evenly distributing the coating in the fold wherein the catheter balloon is rotated about its longitudinal axis in direction of the orifice of the fold.

This method coats or fills specifically the folds and can be performed with any coating solution which is still viscous in such a way that it can be filled into the fold by means of small nozzles or small outlet orifices.

### Spray Method or Fold Spray Method:

This method comprises the following steps:
a) providing a folded compressed catheter balloon,
b) providing a coating device with a plurality of aligned releasing orifices,
c) inserting the plurality of aligned releasing orifices under the fold of the catheter balloon,
d) simultaneous release of a defined amount of a coating solution from the releasing orifices into the fold; and
e) drying of the coating solution in the folds.
   Optionally, step f) for drying can still follow:
f) drying of the coating solution in the fold or evenly distributing the coating in the fold wherein the catheter balloon is rotated about its longitudinal axis in direction of the orifice of the fold.

This method coats or fills specifically the folds and can be performed with any coating solution which is still viscous in such a way that it can be filled into the fold by means of small nozzles or small outlet orifices.

### Drag Method or Drop-Drag Method:

This method comprises the following steps:
a) providing a catheter balloon in a folded, partially inflated or completely inflated state,
b) providing a coating device with a releasing device,
c) forming of a drop of the coating solution at the releasing device,
d) dragging the drop over the surface of the catheter balloon to be coated without the releasing device itself contacting the surface of the catheter balloon, and
e) redosing of the coating solution so that the drop substantially maintains its size.

This elegant and for the catheter balloon particularly careful method uses a drop of the coating solution to be moved or dragged over the surface of the balloon without the releasing device contacting the surface of the balloon, in that the releasing device and thus the drop and the surface of the balloon moving relatively to one another.

The coating solution is thereby redosed in such a way that the drop substantially maintains its size and the connection between the releasing device and the surface of the balloon maintained. By means of a volume measuring device the dispensed amount of coating solution can be exactly determined after the coating and thus the amount of active agent on the balloon.

### Thread Drag Method:

This method comprises the following steps:
a) providing a catheter balloon in a folded, partially inflated or completely inflated state,
b) providing a coating device with a releasing device in the form of a thread, sponge, leather strip or piece of textile,
c) providing a coating solution,
d) soaking the releasing device with the coating solution,
e) transferring the coating solution from the releasing device onto the surface of the catheter balloon to be coated, and
f) redosing of the coating solution so that a consistent release of the coating solution from the releasing device onto the surface of the catheter balloon to be coated occurs.

This likewise very elegant method is also very gentle to the surface of the catheter balloon since the releasing device contacts the surface of the balloon indeed but is developed in such a way that it cannot damage the surface the balloon. The releasing device is pulled or dragged over the surface of the balloon by a movement of the catheter balloon relative to the releasing device and thereby releases a defined amount of coating solution. By means of a volume measuring device it can be precisely determined after the coating, how much coating solution was transferred on the surface of the balloon, thus yielding the exact amount of active agent on the surface of the balloon.

### Ballpoint Method or Roll Method:

This method comprises the following steps:
a) providing a coating device with a ball head for transferring the coating solution onto the surface of the catheter balloon to be coated,
b) providing a coating solution with access to the ball head,
c) setting the ball head of the coating device onto the surface of the catheter balloon to be coated,
d) performing a pressure on the ball head of the coating device for enabling the outflow of the coating solution, and
e) tracing the surface of the catheter balloon to be coated with the ball head thus transferring the coating solution onto the surface of the catheter balloon to be coated.

In this likewise quite elegant method the releasing device rolls over the surface of the balloon due to a movement of the catheter balloon relative to the releasing device and thereby releases by means of a ballpoint an amount of the coating solution onto the surface of the balloon which can be determined with a volume measuring device.

In the following the coating and filling methods according to the present invention are addressed in more detail.

### Pipetting Method or Capillary Method:

In this method a pipette or a syringe or any other device capable of point-shaped release of the composition containing the active agent is used.

The terms “composition containing the active agent” or “coating solution” as used herein relate to the mixture of active agent and solvent and optionally additives, thus a real solution, dispersion, suspension or emulsion of an active agent or combination of active agents, a transport mediator or a mixture of transport mediators and at least one solvent. The term “solution” shall further clarify that it is a fluid mixture which, however, can also be gel-like, viscous or pasty (thick viscous or highly viscous).

The pipette or syringe or outlet or other device capable for point-shaped release of the composition containing the active agent is filled with the composition and its outlet is set preferably at the proximal or at the distal end of a fold. The exiting composition is drawn by capillary forces into the fold and along the fold until the opposite end of the fold is reached.

The catheter balloon is in a compressed, i.e. deflated, state. Even a partial or marginal inflation of the catheter balloon is usually not necessary to slightly open the folds. Nevertheless the filling of the folds can be carried out at a marginal inflation of the catheter balloon up to at most 10% of the diameter planned for dilation. The filling of the folds can also be performed at a slight widening of the folds by applying 100 kPa (1 bar) overpressure, preferably 50 kPa (0.5 bar) for widening slightly the folds.

In this method it is important that the composition containing the active agent is sufficiently thin fluid to develop the corresponding capillary forces.

As compositions solutions of an active agent or of a mixture of active agents and transport mediator or mixture of transport mediators in an alcohol or in a mixture of alcohols are particularly preferred.

The capillary forces should be that strong that a fold with the length of 10 mm is completely filled during 5 to 80 seconds, preferably during 15 to 60 seconds and particularly preferably during 25 to 45 seconds.

If the composition or solution, respectively is too viscous it can be advantageous to incline the catheter balloon with the fold to be filled upwards from the horizontal position to at most 45°, preferably at most 30° and thus also to use gravitation. In general, the filling of a fold by means of capillary forces occurs, however, in a horizontal position of the catheter balloon with the fold to be filled upside. The pipette or syringe or other device capable for point-shaped release of the composition containing the active agent is set onto the fold preferably at the proximal or at the distal end of the fold in a sharp angle in direction of the fold axis in an angle of 10° to 65°, preferably 20° to 55°, more preferably in an angle of 27° to 50° and particularly preferably in an angle of 35° to 45°, measured from the horizontal plane. The filling of the fold is then performed from the upper end of the fold so that the coating solution finds a downhill gradient and additionally to the capillary forces also gravitation is used.

In principle, there is of course also the possibility to set the pipette or syringe or other device capable of point-shaped release of the composition containing the active agent in the middle of the folds or at any other point between the distal and proximal end so that the fold fills itself simultaneously in direction of the proximal and the distal end due to capillary forces, but the starting points at the end of the fold turned out to be preferable.

If the composition for filling the folds or the present fold has reached the opposite end, the substance flow usually stops by itself and the syringe or pipette or the other device capable for point-shaped release of the composition containing the active agent can be removed.

In order to prevent a larger drop of the composition containing the active agent remains at the setting point of the syringe or pipette or the other device capable for point-shaped release of the composition containing the active agent it turned out to be advantageous to already remove the syringe or pipette or the other releasing device before the composition containing the active agent reaches completely the other end of the fold. Thereby the rest of the composition containing the active agent which remained yet at the setting point of the syringe or pipette or the other releasing device is drawn into the fold so that no coating composition, or better filling composition remains outside the fold.

Preferably the syringe or pipette or the other releasing device is removed when ca. 90% of the fold is filled with the composition containing the active agent. The optimal moment for removing the syringe or pipette or the other releasing device can be determined exactly with a few experiments and is also reproducible.

The term “other device capable of point-shaped release of the composition containing the active agent” relates to a device which is similarly to a pipette capable of providing a steady and continuous flow of the composition containing the active agent so that it can also refer to a pump, micro-pump or another reservoir which ensures this steady and continuous release of the composition containing the active agent.

After the filling of a fold the catheter balloon is rotated so that the next fold to be filled lies upside, and preferentially horizontal. The fold filling procedure is now repeated.

Depending on the consistency of the composition containing the active agent it may be necessary to dry the previously filled fold before rotating the balloon for filling the next fold. Drying is preferably performed by evaporation of the solvent.

Further, in this method it is also possible to fill or coat two, more than two or all folds of a catheter balloon at the same time, if the consistency of the composition containing the active agent allows that, i.e. the consistency is not that thin fluid in such a way that the composition leaks out of the folds which are not positioned horizontally.

Particularly the pipetting method is suitable for filling simultaneously several or all folds of a catheter balloon. Herein the catheter balloon can be arranged horizontally or preferably vertically and the releasing devices are set from above to the ends of the folds preferably in an angle of 10 to 70 degrees, so that the composition containing the active agent can flow into the folds.

When all folds of the balloon are filled final drying is performed. In principle, it is not necessary that all folds of the catheter balloon are filled, but the filling of all folds is the common and preferred embodiment, since during dilation a preferably maximum amount of active agent shall be transferred onto the vascular wall in a preferably short time.

In the fold balloons according to the present invention dilation lasts for preferably at most 60 seconds and particularly preferably for at most 30 seconds.

After filling the last fold the last folds are dried, i.e. the content of the last fold preferably without a vacuum under normal pressure by evaporation of the solvent.

To this preliminary drying a final drying can follow which is carried out according to the invention in a rotating catheter balloon. If required or desired, additionally a vacuum can be applied during rotation. This special drying method is described in more detail following the coating methods according to the present invention.

### Squirting Method or Syringe Method:

In this inventive method a fine syringe, syringe-shaped orifice, syringe-shaped outlet or needle or nozzle is set to the proximal or distal end of a fold, and this releasing device in the form of a syringe, needle or nozzle is moved along the longitudinal axis of a fold relatively to the fold and according to the covered section a certain amount of the composition containing the active agent or a defined flow of the coating solution is released.

Herein it is irrelevant whether the catheter balloon is tethered and the releasing device is moved along the fold, or whether the releasing device is fixed and the catheter balloon moves relatively, or whether even both the catheter balloon and the releasing device move towards one another. If the catheter balloon and the releasing device move relatively to each other a movement on a straight line in opposite direction is preferred.

From the releasing device, i.e. the syringe, needle or nozzle or the like, a preferably medium to thick viscous composition containing the active agent is released into the inside of the fold preferably in form of a paste or a gel or an oil. Viscosities of preferred solutions range between 10¹ to 10⁶ mPa·s, preferably between 10² to 10⁵ mPa·s and particularly preferably between 10³ to 10⁴ mPa·s.

Thus especially those compositions containing an active-agent along with the above-listed oily transport mediators such as polyols, phenols, glycerides or alcohols with at least 8 carbon atoms are suitable.

In the coating procedure the tip of the syringe, nozzle or needle reaches ca. up to the middle of the inside of the fold, thus to ca. the middle of the fold, i.e. the nozzle or the outlet is located relatively central in the cavity formed by the fold. There a continuous flow of the composition containing the active agent occurs in such a way that the velocity and the amount of the release in regard of the relative movement velocity of the releasing device and the catheter balloon are suitable to fill the fold the inside of the fold, respectively with the composition containing the active agent by at least 50 volume percent, preferably by at least 70 volume percent and particularly preferred by at least 85 volume percent with the composition containing the active agent.

The filling of a fold lasts at a fold length of 10 mm for ca. 5 to 80 seconds, preferably ca. 15 to 60 seconds and particularly preferably ca. 25 to 45 seconds.

During the filling procedure the catheter balloon is a compressed, i.e. a deflated state. In general even a partial or marginal inflation of the catheter balloon is not required for opening the folds slightly. Nevertheless the filling of the folds can be carried out with a marginal inflation of the catheter balloon up to at most 10% of the diameter planned for dilation. On filling the folds there can be also a slight widening of the folds by applying 100 kPa (1 bar) overpressure, preferably 50 kPa (0.5 bar) for widening the folds slightly.

This coating method can of course also be carried out with fluid compositions containing an active agent, but is rather suitable for oily compositions and also for highly concentrated salt solutions.

Furthermore, this method provides the advantage that more than one fold and particularly all folds can be coated or filled at the same time. Herein a circular array of release devices is arranged according to the number of the folds in such a way that one releasing device per fold is provided. By a slight rotation the tips of the releasing devices are inserted into the folds and placed ca. at the middle of the inside of the folds. By a relative and simultaneous movement of the releasing device relatively to the longitudinal axis of the folds all folds can be filled at the same time with a continuous and steady flow of the composition containing the active agent.

During the filling or coating of one or all folds the catheter balloon may be positioned vertically, horizontally or obliquely.

If volatile solvents have been used in the composition containing the active agent it may be necessary to dry the content of the folds or to remove the volatile solvent with boiling points under 150° C. With volatile solvents this is preferably done first by evaporation of the one or more volatile solvents.

Then a final drying can occur wherein the catheter balloon is rotated in direction of the orifices of the folds, seen from the inside of the folds. This method is addressed more in detail further below. If coating solutions were used that remain oily or pasty after removing the possibly present solvent the rotation drying can serve on the one hand the removing the residuals of the solvent with boiling points below 150° C. and on the other hand the evenly distributing the oily or pasty layers inside the folds.

The turning or rotation of the catheter balloon in direction of the orifices of the folds can also serve to distribute the compositions located in the folds or under the folds inside the respective fold evenly.

This rotation of the fold balloon can be particularly advantageous when using oily or pasty compositions containing an active agent to ensure an even distribution of the composition containing the active agent inside the folds and also on the inner surface of the folds.

The term “coating” as used herein refers preferably also to the coating of the inner surface of the folds, wherein the complete inner space of the fold is generally not filled with the composition containing the active agent or the composition remaining after drying, respectively.

In contrast, the term “filling” rather relates to a complete filling of the inner space of the folds with a composition containing an active agent.

If solvents are used which can be removed by drying in general a filling cannot be obtained and it is rather referred to as a coating of the inner surfaces of the folds.

If however substances with a high boiling point are used as additives, such as e.g. oils, a more or less complete filling of the folds is possible as long as no considerable amount of volatile substances is present in the composition containing an active agent. The use of additives is, however, optional.

This squirting method or syringe method is particularly suitable for the application of compositions containing an active agent into the folds of fold catheter balloons which cannot be applied onto a catheter balloon, let alone inside the folds by conventional dipping and spraying methods.

In contrast to the conventionally used solid coatings on stents or on catheter balloons these oily and pasty coatings and fillings have the advantage that these compositions containing an active agent do not dry completely but maintain their consistency to a large extent. Thus coating solutions are preferably used which do not harden completely on air or under an inert-gas atmosphere at normal pressure, i.e. after extensive removing of a possibly used solvent of the coating solution an oily or pasty coating remains inside the folds of the catheter balloon after the solvent was removed by evaporation or under reduced pressure. Thus coating solutions are preferred which after removing the optionally used solvent have a melting point or solidification point of less than 20° C., preferably less than 30° C. and additionally display a thick viscous, oily or pasty consistency in order that also when storing the coated catheter balloon for several months up to one year the coating does not ooze out of the folds.

The use of a solvent which has to be removed is, however, not compulsory, so that also physiologically acceptable solvent can be used, such as polyethylene glycol, glycerine, propylene glycol or the like, which will not be removed and remains in the coating and keeps the coating in the folds oily and pasty for the shelf life of the coated medical product.

The enormous advantages of such oily and pasty coatings are evident. If the catheter balloon is inflated or dilated at the stenotic place this oily and pasty composition is at least partially, but in general to a large extent transferred onto the vascular wall and serves as an active agent reservoir for a delayed release of the active agent to the adjacent tissue for several hours up to days and additionally has the benefit of dissolving plaques or counteracting the sedimentation of plaques, respectively, and is biologically degraded itself later on without releasing physiologically critical metabolites. This system perfectly solves the problem to apply on the one hand a coating safely to the catheter balloon in such a way that it is not washed away by the bloodstream when being introduced or not transferred when contacting the vascular wall, and on the other hand that a sufficient amount of the active agent is transferred during dilation onto the vascular wall in a relatively short time, i.e. in 30 to 300 seconds, i.e. as less as possible coating remains on the catheter balloon and as much as possible, i.e. at least 50% of the coating is transferred onto the vascular wall, for effectively counteracting restenosis.

Such systems according to the invention cannot only be produced by the squirting method, but also by the other coating methods described herein.

### Spray Method or Fold Spray Method:

In this method according to the invention a plurality of aligned release orifices is moved or set under the fold of the fold balloon and a composition containing an active agent is released simultaneously from the plurality of apertures into the respective fold.

The release device consists preferably of 2 to 10 nozzles or release orifices which are aligned at preferably equal intervals along the longitudinal direction of the folds.

This release device is then inserted under the fold of the catheter balloon and the respective fold is filled or coated by simultaneous release of the composition containing an active agent from the nozzles or other release orifices.

Similar to the aforementioned squirting method the filling of a fold lasts ca. 5 to 80 seconds, preferably ca. 15 to 60 seconds and particularly preferably ca. 25 to 45 seconds when having a fold length of 10 mm and using 4 release orifices. The release orifices are preferably located mainly in the middle of the cavity under the folds.

In this coating or filling variant it is not necessary to move the release device in the fold of the catheter balloon relatively to the longitudinal direction of the fold. In general the catheter balloon and the release device are fixed during the filling or coating wherein, however, a movement along the longitudinal direction of the fold is possible. If a relative movement is planned, then the distance for the movement is preferably not larger than the distance between two nozzles or release orifices of the release device.

The release device comprises or consists of at least 2 and at most 10 release orifices or nozzles or the like, and preferably of 3 to 6 and particularly preferably of 4 or 5 release orifices or nozzles or the like, being preferably evenly distributed over the distance of 10 mm.

The release device has 2 to 10 nozzles or similar orifices being capable of releasing the composition containing an active agent evenly or of spraying evenly into the fold.

For this filling or coating method preferably medium to thin viscous compositions or solutions of an active agent or of a combination of active agents are used which notably contain an alcoholic solvent. Further, coating solutions are preferred which do not harden completely but maintain a gel-like, viscous, oily or pasty consistency. Here also the above statements on the squirting method apply, especially for the coating solution and drying.

In this fold spray method the catheter balloon is in a compressed, i.e. deflated state. Even a partial or marginal inflation of the catheter balloon is usually not required to open the folds slightly. Nevertheless the filling of the folds can be carried out with a marginal inflation of the catheter balloon up to at most 10% of the diameter planned for dilation. Filling the folds can be also performed at a slight widening of the folds by applying 100 kPa (1 bar) overpressure, preferably 50 kPa (0.5 bar) for widening the folds slightly.

After filling a fold the catheter balloon is rotated so that the next fold to be filled lies preferably upside and preferably horizontally. The fold filling or fold coating procedure will now be repeated.

Depending on the consistency of the composition containing an active agent it may be necessary to dry the previously filled fold before rotating the balloon for filling the next fold. The drying is preferably performed by evaporation of the solvent.

Furthermore it is also possible in this method to coat or fill simultaneously two, more than two or all folds of a catheter balloon if the consistency of the composition containing an active agent allows that, i.e. if the consistency is not that thin fluid that the composition leaks out of the folds which do not lie horizontally. For filling or coating several or all folds an corresponding circular disposition of release devices according to the number of folds is provided and placed around the preferably vertically oriented catheter balloon, and by rotation the release orifices are directed under the folds where the simultaneous release of the composition containing an active agent occurs.

When all folds of the balloon are filled final drying occurs. Basically it is of course not necessary to fill all folds of the fold catheter balloon whereas the filling of all folds, however, is the common and preferred embodiment, since during dilation a preferably maximum amount of active agent shall be transferred onto the vascular wall in a minimum short time.

After filling of the last fold the drying of the last folds occurs, i.e. of the content of the last fold preferably without vacuum under normal pressure by evaporation of the solvent.

To this preliminary drying a final drying can follow which according to the invention is carried out on a rotating catheter balloon. If required or desired, a vacuum can be applied additionally during rotation. This special drying method is described in more detail in the following of the coating methods according to the invention.

### Drag Method or Drop-Drag Method:

A particularly preferred method for the overall coating as well as for specific coating or filling of the folds is the so-called drag method or drop-drag method.

This method allows coating of a catheter balloon in its compressed state with a fluid composition containing an active agent over the complete area inside and outside the folding.

In this method a releasing device in the form of a syringe, needle, pipette or nozzle is approached to a preferably horizontally tethered, fix or preferably rotating balloon and then a volume of the composition containing an active agent is dosed in such a way that at the tip of the releasing device a drop is formed which contacts the dosing device as well as the balloon.

For a better performance the dosing device can preferably be prolonged at the releasing end with a thin wire, thread or spongiform auxiliary tool that upon approach the liquid contact between the dosing device and the balloon is established and maintained via this auxiliary tool.

Optionally there is also the possibility to use a dosage needle with a lateral orifice or a fork-shaped extension.

By a lateral movement of the dosing device along the longitudinal direction of the balloon relative to the rotating balloon the drop is dragged and a certain amount of the composition containing an active agent dries as a thin film on the traced surface per covered section. Herein the drop size is maintained by re-dosing the composition containing an active agent until the final dosage is reached.

The movement is maintained as long as the complete target surface is coated and no liquid is present anymore on the balloon surface.

In order to counteract the capillary effect of the folding at the initial dose serving for building a drop between the balloon surface and the dosing device the balloon can be previously wetted with a suitable solvent, because then the folds are already filled with liquid and the capillary effect does not suck up the drop or may potentially improve the better grip of the filling material on the material of the balloon, respectively.

As most of the tips of releasing devices are made of harder or hard materials, or of a material being able of damaging the balloon material which may lead to perilous complications during dilation, a particularly preferred embodiment consists in conducting or tethering a thread or wire at the tip of the releasing device or through the releasing device or at least the terminal orifice of the releasing device, which then serves for contacting to the balloon surface without the tip of the releasing device contacting the balloon. This thread or wire consists of a material which cannot damage the balloon material.

Instead of a thread or wire also a sponge or spongiform matter, a piece of textile or a correspondingly thin dimensioned piece of leather, or a bunch of hair or bristles can be used. It is required, however, that these tools consist of materials that do not damage the catheter balloon, i.e. they are not sharp or edged, nor release corrosive, basic, acid or sticky substances or chemicals which could dissolve completely or partially, decompose, stiffen, scratch or cut the polymer of the catheter balloon.

Thus particularly such substances and polymers are preferred as materials for these tools from which also textiles, threads, yarns, bristles for brushes can be manufactured.

According to the invention it is thus achieved that the tip of the releasing device can be held at a certain distance to the balloon surface and yet the drop and the movement of the drop relative to the balloon surface can be controlled and regulated via the contacting device in the form of a thread, wire, sponge, leather strip, bristle or piece of textile.

Basically it does not matter whether the releasing device is moved with the balloon being stationary or the balloon is moved with a stationary releasing device. A preferred embodiment consists of a rotating balloon in a horizontal position together with a releasing device arranged from above and moving along the longitudinal axis of the balloon. In this embodiment a spiral coating of the complete surface of the catheter balloon occurs.

In another preferred embodiment the coating of the catheter balloon in a horizontal position occurs at intervals. With the balloon being stationary the releasing device moves along the longitudinal direction of the catheter balloon in an approximately straight line from one end to the other and back, wherein the balloon is rotated about some degrees when the releasing device reaches the distal or proximal end of the catheter balloon. A line-shaped coating of the complete balloon surface occurs through this embodiment.

If the releasing device is set however on a fold and it is moved along the fold and this procedure is repeated with the other folds after rotating the balloon a specifically fold-filled catheter balloon results.

### Thread Drag Method:

In this method no drop is moved over the surface of the catheter balloon but a thread connected with the releasing device, or serving as a releasing device, is dragged over the surface of the balloon or set or stippled onto the balloon surface and can serve also in the inoperative state for releasing a solution containing an active agent.

In this procedure a solution containing an active agent flows along the thread wherein preferably no drop formation occurs. The thread is permanently wetted with the solution containing an active agent and releases this solution to the balloon surface as soon as the thread gets in contact with it.

Also this method has the big advantage that the tip of the releasing device consisting mostly of a hard material does not touch the balloon material, similar as in the drop-drag method, and thus no damaging of the catheter balloon occurs.

Preferably, the thread is dragged horizontally along the longitudinal direction while the catheter balloon is rotating, wherein it releases a rapidly drying trace of solution containing an active agent.

This method, however, is not limited to an embodiment with a thread, but also several threads can be moved simultaneously over the balloon surface, wherein in this case the balloon is preferably vertically positioned. Moreover, the threads can also be linked or form a mesh. Herein the threads are linked with at least one releasing device which continuously supplies the threads or the mesh with a solution containing an active agent.

This method thus is suitable for the complete or partial coating of the balloon surface. If only the folds should be filled or coated instead there is the option of inserting a thread at least partially into the fold, or to place it into the fold when folding the balloon, and let the solution containing an active agent flow into the fold via this thread, wherein after filling the fold the thread is preferably removed.

Further, for the specific filling of the folds a combination of the pipetting and the thread drag method is particularly suitable, wherein such a big amount of the solution containing an active agent is released from the releasing device by means of the thread at the proximal or distal end into the unfilled fold of an inflated catheter balloon that the capillary effect sucks the solution into the fold.

The drop-drag method as well as the thread drag method both solve elegantly the problem to coat or fill specifically the balloon surface as well as specifically the folds of the balloon with a defined amount of active agent without damaging the balloon material. The releasing device may have a volume measuring device which records or displays the released amount of solution containing an active agent.

Further, these methods are particularly suitable for coating and/or filling the folds of a balloon in the deflated (folded) state which is particularly demanding since the balloon surface of a folded balloon is not formed evenly and the common coating methods for regular-shaped bodies can only be applied with the corresponding problems. However, in the drop-drag method or thread drag method differences in distance between the balloon surface and the releasing device are compensated elegantly by the contacting device in the form of a thread, wire, sponge, leather strip, bristle, or piece of textile.

### Ballpoint Method or Roll Method:

A preferred variation of the drop-drag method consists of using a coating head, which is ball shaped. The ball has such a diameter that it just cannot drop out of the outlet orifice of the coating container. It shuts the container completely so that no coating solution can exit between the ball and the vascular wall. When pressure is applied on this ball when contacting the object to be coated the ball moves into the container according to the variably applied pressure and the coating solution can exit between the ball and the vascular wall of the solution container. With a simultaneous movement of either the coating container or of the object to be coated and a desired angle between them the ball rolls on the surface and ensures a particularly even coating of the surface. This way the various objects can be coated within the given shape since the ball can trace the surface like a sensor by means of the adjustable pressure and angle and thus provides a particularly high variability in respect to the surfaces to be coated and also to the coating options.

This coating method can be applied excellently especially in catheter balloons since each catheter balloon has a different surface design, is uneven and no balloon surface is equal to another. A preferably optically controlled ballpoint coating method offers the option of coating any different and uneven as well as unequal surface evenly. Further, the ball head for transferring the coating solution has the advantage that it does not damage the surface of the catheter balloon and the ball head or the ball, respectively can be manufactured of a soft or rubber-like material such as e.g. natural rubber, silicon or polymers of similar consistency which is even more gentle to the balloon surface in comparison with a metal ball.

Since the ball head can be placed very precisely there are controlled starting and end points for the coating. Further, the coating device can be designed in such a way that a three-dimensional movement is possible so that the entire catheter balloon can be coated without even once setting off or resetting the ball head. After tracking the balloon surface to be coated in a serpentine way the ball head of the coating device gets back to the starting point, wherein the initially coated tracks have dried in the meanwhile and a further coating layer can be applied onto the first, by which coating and drying processes can be carried out without interruption of the entire coating process.

Furthermore a well controllable and even coating results from the roll movement of the ball head, wherein the thickness of the coating layer can be controlled via the pressure applied to the ball and the speed of the motion.

### Rotation Drying:

As mentioned above the coated or filled catheter balloons can be dried during rotation after coating or filling each fold or after coating or filling all folds or of the folds to be coated or filled if not all folds shall be coated or filled. This is most of the times indicated as step f) in the methods according to the invention.

This rotation drying has several advantages. On the one hand the composition containing the active agent is dried and additionally evenly distributed inside the folds as well as on the surface inside the folds.

The rotation drying is particularly suitable for oily or viscous compositions containing an active agent in order to obtain an even distribution of the composition in the respective fold, wherein these coatings in general become not dry but maintain their viscous, oily, gel-like or pasty consistency which is also desired and particularly preferred.

Additionally vacuum can be applied during the rotation of the catheter balloon in order to obtain an intensive drying of the composition containing an active agent.

During drying under vacuum especially in viscous, high viscous or solidifying solutions boiling delays occur, i.e. residuals of the solvent pocketed in the oil or solid are released spontaneously and tear or bust the coating or filling. Upon drying under vacuum with a simultaneous rotation these boiling delays are avoided and a dried and/or oily, viscous, gel-like or pasty even coating of the inner surface of folds is obtained.

Moreover, the sense of rotation is crucial. The sense of rotation is in direction of the orifices of the folds when regarding them from the inside of the fold. The catheter balloon is thus rotated like the bucket wheel of a bucket-wheel excavator in order of pressing the composition containing the active agent into the inside of the folds by means of the rotatory force.

Preferably the fold balloon is rotated with a rotatory velocity of 50 to 500, preferably 150 to 300 rotations per minute.

Depending on the active agent to be imported into the folds or depending on the consistency of the composition containing the active agent to be imported under the folds of a catheter balloon the suitable coating method according to the present invention can be selected.

All coating methods according to the invention which enable a specific coating or filling of the folds are suitable, optionally together with a rotation drying method, for enabling a non-solid but oily, gel-like, pasty or high viscous coating or filling of the folds.

The fold spray method is preferably suitable for thin to medium viscous compositions containing an active agent, while the pipetting method is preferably suitable for light, medium and slightly hard viscous compositions and the squirting method is particularly well applicable for medium viscous, viscous to high viscous compositions.

The term viscosity refers to the dynamic viscosity [η]:

$$[ η ] = \frac{kg}{m \cdot s} = Pa \cdot s = \frac{Ns}{{m}^{2}}$$

The squirting method can be preferably used for thick viscous compositions. Preferred are viscosities at room temperature in the range of oils (olive oil: 10² mPa·s), honey (10³ mPa·s), glycerol (1480 mPa·s) or syrup (10⁵ mPa·s). This method works of course also with thin viscous solutions with η≦10² mPa·s.

The pipetting method can be used preferably in medium viscous solutions. Preferred are viscosities at room temperature in the range of 0.5 mPa·s to 5000 mPa·s, more preferred in the range of 0.7 mPa·s to 1000 mPa·s, even more preferred in the range of 0.9 mPa·s to 200 mPa·s and particularly preferred in the range of 1.0 mPa·s to 100 mPa·s. In this viscosity range oils, contrast media and/or salts can be found which are diluted with common solvents, especially alcohols. The pipetting method can be applied over a very broad viscosity range.

The fold spray method is preferably used in thin viscous compositions. Preferred are viscosities at room temperature in the range of 0.1 mPa·s to 400 mPa·s, more preferred in the range of 0.2 mPa·s to 100 mPa·s and particularly preferred in the range of 0.3 mPa·s to 50 mPa·s (water: 1.0 mPa·s; kerosene: 0.65 mPa·s; pentane: 0.22 mPa·s; hexane: 0.32 mPa·s; heptane: 0.41 mPa·s; octane: 0.54 mPa·s; nonane: 0.71 mPa·s; chloroform: 0.56 mPa·s; ethanol: 1.2 mPa·s; propanol: 2.3 mPa·s; isopropanol: 2.43 mPa·s; isobutanol: 3.95 mPa·s; isotridecanol: 42 V mPa·s).

### Coated Catheter Balloons

According to the methods disclosed herein catheter balloons without a stent and partially also with a stent can be coated so that the present invention relates to coated catheter balloons which can be obtained by the methods described herein.

A particularly preferred embodiment uses a catheter balloon with a crimped stent. This stent can be a non-coated (bare) stents or preferably a stent coated with only one hemocompatible layer. As hemocompatible layer particularly are preferred the heparin and chitosan derivatives disclosed herein and primarily desulfated and reacetylated or re-proprionylated heparin.

Moreover, there is the option of applying under and/or on the layer containing the transport mediator yet one or more layers of pure a active agent or a polymer or polymer containing an active agent.

Upon using the fold balloons which form folds when being compressed these can be filled with active agent and transport mediator. Particularly the pipetting method is suitable therefore.

A possibly used solvent can be removed under reduced pressure, the mixture inside the folds can thus be dried. On dilating such a balloon which in general is used without a stent the folds turn or bulge to the outside and thus release their content to the vascular wall.

The methods according to the invention are not only suitable for the coating of catheter balloons but also for the coating of guide wires, spirals, catheters, cannulae, tubes and generally tubular implants or parts of the aforementioned medical products if a structural element comparable to a stent is contained in such a medical product that shall be coated or filled. Vascular supports and especially stents such as coronary, vascular, trachea, bronchia, urethra, oesophagus, gall, kidney, small intestine, colon stents for example can be coated.

The coated medical devices are particularly used for keeping all duct-like structures open, for example of the urinary tract, oesophagus, trachea, bile duct, renal tract, blood vessels in the whole body including the brain, duodenum, pylorus, small and large intestine, but also for keeping open artificial outlets, as being used for the intestine or the trachea.

Thus the coated medical devices are suitable for the prevention, reduction or treatment of stenoses, restenoses, atherosclerosis, atherosclerosis, and all other forms of occluded vessels or stenoses of passages or outlets.

The balloon catheters according to the invention without a stent are particularly suitable for the treatment of in-stent restenosis, i.e. for the treatment of recurring vessel stenoses inside an already implanted stent which preferably is not bioresorbable. In such in-stent restenoses the placement of another stent inside the already existing stent is particularly problematic as the vessel in general can only insufficiently be widened by the second stent. Herein the application of an active agent by means of balloon dilation offers an ideal treatment method since this treatment can be repeated several times, if necessary, and from a therapeutic point of view may obtain the same or significantly better results than another stent implantation.

Furthermore the inventive catheter balloons without a crimped stent are particularly suitable for the treatment of small vessels, preferably small blood vessels. Small vessels refer to those vessels with a vessel diameter less than 2.5 mm, preferably less than 2.2 mm.

In summary the following applies for the use of the selected additives and excipients:

The abovementioned additives and excipients as well as their mixtures and combinations preferably have at least one of the following characteristics for successful local application of one or more active agents:
1) the exposure time of the short-term implant is sufficient for the transfer of a suitable therapeutic amount of the active agent into the cells,
2) during exposure a sufficient amount of coating material containing the active agent adheres to the vascular wall for ensuring the desired therapeutic effect, and it is particularly preferred
3) that the coating containing the active agent being present on the short-term implant has a higher affinity to the vascular wall than to the surface of the implant so that an optimal transfer of the active agent onto the target can occur. This works very well mainly for pasty, gel-like or oily coatings.

Of course in all cases a coated or uncoated stent can form a system with the balloon catheter, depending on the individual requirements. Likewise other excipients such as the imaging agents can be added, if required.

For example, the exposure time of the particularly preferred embodiment of a balloon catheter with paclitaxel coated by the spray method is already sufficient for applying a therapeutic amount of paclitaxel being sedimented amorphously by way of the spray method together with the at least one transport mediator onto and into the cell wall. Here, a stent rendered hemocompatible with a semi-synthetic oligosaccharide and likewise coated with paclitaxel serves as a reservoir for the elution of further amounts of active agent planned for a longer time span.

Because of the amorphous consistency of paclitaxel on the stent and the catheter balloon obtained from the special spray method paclitaxel is not flushed or washed off the surface during the introduction of the catheter so that the desired amount of active agent reaches its target site and is released here by dilation to the vascular wall. Because of the simultaneous coating of the stent and the catheter balloon the vessel is additionally completely covered with active agent. Further it is preferred when the catheter balloon is also coated with paclitaxel in the sections extending the stent ends so that a supply of the vessel with paclitaxel (or any other active agent instead of paclitaxel) occurs also in the section of the stent ends and beyond for 1 to 3 mm in proximal and distal direction. Also here the amorphous structure of paclitaxel is of particular importance because only thereby the surface of the layer with the active agent is enlarged in such a way that an optimal amount of the active agent adheres to the cell wall and can enter the cell wall or the cells, respectively.

The addition of a vasodilator directly acting on the cell wall or of a carrier easily permeating the membrane (e.g. DMSO, PETN, lecithin) can still enhance significantly the uptake into the cells during the accumulated exposure time of preferably 30 to 300 seconds.

In another particularly preferred embodiment of substance-eluting balloon catheter the active agent is dissolved together with a hydrophobic long-chained fatty acid, e.g. glycerol monooleate, in a suitable solvent and applied to the surface of the catheter balloon. For coating all coating methods described in the following are suitable. The addition of the glycerol ester enables the transfer of the coating material from the surface of the catheter onto the vascular wall, wherein the amount of the transferred substance-eluting matrix is sufficient to provide the active agent in a sufficient concentration as well as to prevent the coating from being instantaneously washed away in the blood stream.

A further particularly preferred embodiment consists in the use of mixture with high affinity to the cell wall of the polysaccharide carrageenan, phosphatidylcholine, one of the major components of cell membranes, as a membrane-permeating substance and glycerol that due to its very good adhesive properties allows a delayed release of the active agent of up to 12 hours after dilating the vessel. All coating methods are suitable for this embodiment, particularly preferred are the pipetting, thread drag and ballpoint method described herein.

### EXAMPLES

### Example 1

### Preparation of a Solution of Active Agent and Transport Mediator on the Example of Coniferyl Alcohol and Paclitaxel.

Depending on the consistency also higher concentrations of the active agent become necessary or are desired because of the desired effectiveness.

### A. Coniferyl Alcohol and Paclitaxel

a) ratio of active agent to transport mediator: 9/1. 2 mg of coniferyl alcohol are dissolved in 0.5 μl acetone. 18 mg paclitaxel are dissolved in 0.5 μl acetone as well. Both solution are mixed with each other and can now be used as coating solution.
b) ratio of active agent to transport mediator: 7/3. 6 mg of coniferyl alcohol are dissolved in 0.5 μl acetone. 14 mg paclitaxel are dissolved in 0.5 μl acetone, as well. Both solution are mixed with each other and can now be used as coating solution.
a) ratio of active agent to transport mediator: 5/5. 10 mg of coniferyl alcohol are dissolved in 0.5 μl acetone. 10 mg paclitaxel are solved in 0.5 μl acetone, as well. Both solution are mixed with each other and can now be used as coating solution.
B. Ascorbyl palmitate and cyclosporine A. The procedure is carried out analogous to 1A.
C. Vanillin and rapamycin. The procedure is carried out analogous to 1A. Ethanol is used as a solvent.
D. Curcumin (Active agent list) and paclitaxel. Curcumin and paclitaxel are dissolved in chloroform. The procedure is carried out as in 1A.
E. Farnesol and epothilone. Farnesol and epothilone are dissolved in ethanol as described in Example 1A.
F. Ferulic acid and paclitaxel. The procedure is carried out as in example 1A.
G. Octyl phenol ethoxylate and trapidil. The procedure is carried out as in 1A, but methanol is used as a solvent.
H. Borneol or camphor and rapamycin. The procedure is carried out as in 1A. Chloroform is used as a solvent.
I. Bisabolene and paclitaxel. The procedure is carried out as in example 1A.
J. Ocimene, myrcene or phellandrene (isomers) and cyclosporine A. The procedure is carried out as in 1A. Chloroform is used as a solvent.
K. Linalool and everolimus. The procedure is carried out as in example 1A.
L. β-santalene and kamebakaurin. The procedure is carried out as in example 1A.
M. Squalene and doxetaxel. The procedure is carried out as in example 1A. Chloroform is used as a solvent.
N. Zeaxanthin or/and its isomeric lutein and pimocrolimus. The procedure is carried out as in example 1A.
O. Fosfestrol (synthetic estrogen). Fosfestrol is an antineoplastic active agent and at the same time a transport mediator. Therefore, fosfestrol can be used also without any further additive. For this, for instance for a 4% solution, 20 mg fosfestrol are dissolved in 1 ml ethanol.
P. Fosfestrol and rapamycin. With the main function of fosfestrol as transport mediator a combination of the active agent effects can be obtained. The procedure is carried out as in example 1A.
Q. Lycopene and tacrolimus. The procedure is carried out as in example 1A. Methanol is used as a solvent.
R. Tibolone and paclitaxel. The procedure is carried out as in example 1A.
S. Ascorbic acid ether and rapamycin. 0.5 mg of ascorbic acid ether is dissolved in 0.5 μl chloroform and then combined with a solution of 19.5 mg rapamycin in 0.5 μl chloroform.
T. Fumaric acid ether and zotarolimus. The procedure is carried out as in example 1A.
U. 1,8-cineol and paclitaxel. The procedure is carried out as in example 1A.
V. Benzethonium chloride and fasudil. 10 mg benzethonium chloride are dissolved in 0.5 μl ethanol/water (50/50 v:v). 20 mg fasudil are dissolved in 0.5 μl bidistilled water. Both solutions are combined.

### Example 2

Coating of a balloon in two steps with coniferyl alcohol and rapamycin in ratio 9:1 (weight-%) and 5:5 according to examples 1Aa and c)

The thin viscous mixture of example 1Ac is first applied to the catheter balloon in compressed state via the dipping method. Therefore the balloon is dipped vertically into the dipping solution and pulled out again vertically out of the solution that slowly (v<1 mm/s) that an even, bubble-free film can be formed on the surface of the catheter.

After a short drying time of at most 30 minutes the folds are filled again specifically with the coating solution of Example 1Aa) by the pipetting method to guarantee a complete coating and optimal loading of the balloon catheter. For this the coated balloon catheter is arranged on a rotation motor with an angle of inclination of 25° in such a way that the balloon catheter cannot become bent. The dosing syringe which ends in a blunt cannula will be positioned in such a way that it can be introduced that way into the fold from the upper end of the fold and a defined amount of the coating solution can be given into the fold.

After filling of the fold the balloon catheter is rotated around its longitudinal axis after a waiting time of up to 30 s, so that the next fold can be filled.

With the help of the angle of inclination the capillary action and gravity can be used to fill the fold completely or partially depending on the desired dosing.

### Example 3a

The complete and even coating of the folds is possible by installing the balloon catheter at the rotation motor in such a way that it is tethered horizontally and without bending or sagging. The fold to be coated is lying atop, so that it cannot slip away sideways.

Now the coating cannula is positioned in such a way that it grips the fold when moving from the proximal to the distal end of the fold and the other way around, so that only this part of the material of the fold moves up which is filled with coating solution upon movement of the cannula along of the fold at the same time.

That way an even distribution of the coating solution from the start of the fold to the end of the fold is obtained.

The speed the cannula is moving along the fold horizontally and the penetration depth into the fold are set in such a way that the fold closes evenly after the filling step.

The drying of the balloon catheter being coated this way is carried out by rotation drying at room temperature.

A catheter balloon is coated with a biostable coating of cellulose nitrate by the drop-drag method.

For this purpose the catheter is fixed into the adapter of the rotation motor in such a way that it is tethered in a horizontal position without a bending or sagging being possible. The releasing device is tethered over the balloon in such a way that the distance of the pipette through which the coating solution exits is precisely that long that the exiting drop contacts the surface of the balloon without detaching from the pipette tip. The velocity by which the coating solution exits is adjusted in such a way that the drop cannot pull off during the longitudinal movement of the catheter balloon. When the surface of the balloon lying atop is coated completely in such a way the balloon is rotated that far that the adjacent sector can be coated in the same longitudinal direction. The procedure is as often repeated until the balloon catheter has performed a complete cycle.

### Example 3b

On this layer a coating solution according to Examples 1A-V or also a mixture of such coating solutions can be applied onto the balloon.

### Example 3c

On this layer a pure layer of active agent made of paclitaxel is applied.

If necessary the layer of active agent made of paclitaxel may be coated with a barrier layer of polylactides, polyglycolides, polyanhydrides, poyphosphazenes, polyorthoesters, polysaccharides, polynucleotides, polypeptides, polyolefins, vinylchloride polymers, fluorine-containing polymers, teflon, polyvinyl acetates, polyvinylalcohols, polyvinylacetals, polyacrylates, polymethacrylates, polystyrene, polyamides, polyimides, polyacetals, polycarbonates, polyesters, polyurethanes, polyisocyanates, polysilicones as well as co-polymers and mixtures of these polymers.

### Example 4

The balloon catheter is coated completely with an alcoholic solution of a meristyl alcohol and paclitaxel (or another active agent or combination of active agents) via the thread drag method.

Therefore, a 2% solution of meristyl alcohol is prepared in which such an amount of paclitaxel is dissolved that a 30% (weight %) solution of the active agent is derived.

The balloon is coated completely with this solution and then dried under slow rotation about the longitudinal axis at room temperature for at least three hours. This procedure is repeated at least one time.

After complete drying the balloon catheter coated in such a way with active agent can be coated with a 1% PVA solution, for example with a topcoat, in the same way or by another suitable method such as the roll method.

### Example 5a

The fold balloon expanded to nominal pressure is dipped into a 1% dipping solution of paclitaxel and chloroform for 5-10 s and subsequently dried under rotation about the longitudinal axis to such a degree that the major portion of the chloroform has evaporated. Before a complete drying the balloon is deflated again in the air stream. A transport mediator can be optionally added to the paclitaxel solution.

### Example 5b

The fold balloon is tethered in a horizontal position on the rotatable axis so that the fold to be filled is always lying upside. Thus step by step each fold is filled with a solution containing an active agent (e.g. from example 17) which displays a honey- or syrup-like viscosity (viscosities from 10² to 10⁵ mPa·s) from the beginning to the end of the fold by means of a teflon cannula as extension of a needle syringe.

Therefore, the teflon cannula is conducted to the centre of the cavity formed by the fold, and during the movement of the horizontally tethered catheter in its longitudinal direction a defined amount of a high viscous solution is released into the fold cavity (squirting method). The amount of the filled material is limited in such a way that after filling the fold doesn't lift from the balloon body and varies corresponding to different balloon dimensions and manufacturers.

### Example 5c

The balloon loaded with active agent and re-deflated in Example 5a like the fold balloon from Example 5b partially loaded with active agent, can be coated in a second step through the spray method with a polymeric outer layer as a barrier. Therefore, the concentration of the polymeric spray solution must be kept so low that the polymeric layer obtained after drying does not hamper a regular unfolding. For example, a 0.5% PVP solution is already suitable here. A transport mediator according to the list on page 22 is optionally added to the polymer solution.

### Example 6

A catheter balloon is coated with a layer of active agent of paclitaxel and transport mediator. Then the catheter balloon is provided with a protective cover for preventing the active agent of premature detachment, as used in self-expanding nitinol stents. The protective cover can be removed in vivo immediately before dilation.

### Example 7a

A solution of desulfated heparin is prepared in a methanol/ethanol mixture and acidified with acetic acid so that a pH value of 3 to 5 results. Paclitaxel is added to this solution. A catheter balloon is coated with this solution and subsequently a slight cross-linking of the dried coating on the balloon with glutaraldehyde is carried out.

### Example 7b

In a second coating step a transport mediator solution with or without active agent according to Example 1 is applied.

### Example 8

Paclitaxel is dissolved in DMSO containing ca. 10 vol. % water. Potassium oxalate, sodium chloride, glutaminic acid and oxalic acid are added to this solution and the catheter balloon is coated several times with this solution by using the thread drag method and dried after every coating sequence. Subsequently, the coated catheter balloon is provided with a biodegradable layer of a lactam. A transport mediator according to page 22 can be added in both layers or in either one or the other layer.

### Example 9

Paclitaxel is mixed with magnesium sulfate, potassium chloride, lithium chloride and sodium acetate and worked up with a transport mediator to a paste by adding an alcoholic solvent, which then is filled into a syringe and is squirted under the folds of a fold balloon. During coating the tip of the squirting nozzle traces along the fold and applies a layer of paste in the fold along the longitudinal direction of the fold.

### Example 10

A thin viscous ethanolic solution of paclitaxel is prepared which is so thin viscous that the solution is dragged into the folds by itself through capillary forces. By means of a capillary set on one end of the fold the alcoholic paclitaxel solution is let to flow into the fold until the inner space of the fold is filled completely by capillary forces. The content of the fold is left for drying, the balloon is rotated and the next fold is filled. Each fold is filled only once. For this purposes 100 μg benzethonium chloride per ml ethanol is used as a transport mediator.

### Example 11

A mixture of 70% linseed oil and 30% olive oil is prepared. This mixture is dissolved in a ratio of 1:1 in chloroform and after adding paclitaxel (25 weight-%) and ocimen (2 volume-%) applied onto an evenly rotating catheter balloon by means of the roll method. After evaporating the chloroform in a gentle airstream the balloon catheter is stored in a drying closet at 70° C. so that a surface is provided which is already adhesive but soft, highly viscous and thus not impeding on expanding the balloon.

### Example 12

A cobalt/chromium stent is crimped onto a catheter balloon of polyamide.

Thereafter a solution of paclitaxel and a transport mediator in DMSO is applied onto the stent by means of a syringe. The solution is so thin viscous that it flows between the closely fitting struts of the stent and fills the interspaces between the balloon surface and the inner surface of the stent as well as between the single struts of the stent. The solvent evaporates and the pure active agent sediments as a solid onto the catheter balloon under the stent, into the stent interspaces and on the stent and the balloon surface. The catheter balloon is coated with active agent at both ends of the stent for ca. 2 to 3 mm beyond the stent end.

### Example 13

A solution of rapamycin in ethanol is prepared and the solution is sprayed several times on a catheter balloon without a stent, and the catheter balloon is dried in the meantime by letting the solvent evaporate.

After repeating the spray coating three times, wherein in the last step of the spray coating the transport mediator linalool is present in the coating solution, the catheter balloon is finally dried and an uncoated metal stent is crimped onto the balloon.

### Example 14

A commercially available catheter balloon is coated with an amount of 3 μg paclitaxel per mm² balloon surface. The coating is done with the pipetting method by using a solution of paclitaxel in acetone and a transport mediator according to the selection at page 22. Then an uncoated cobalt/chromium metal stent is crimped onto the coated catheter balloon.

### Example 15

A catheter balloon with a crimped uncoated metal stent is coated with a solution of paclitaxel and papain in DMSO by means of the drop-drag method. The coating procedure is repeated three to four times until the interspaces between the balloon surface and the inner surface of the stent as well as the interspaces of the single struts of the stent are visibly filled with active agent.

If desired, a protective layer of for example a polylactide can be applied additionally onto the layer with the active agent paclitaxel.

### Example 16

A commercially available catheter balloon is coated with a dispersion of paclitaxel and maltitol in ethyl acetate with 5 vol. % acetic acid so that an amount of 2-3 μg paclitaxel and 0.1 μg 0.2 μg maltitol per mm² balloon surface results. A bioresorbable stent of polyhydroxybutyrate is crimped onto the coated balloon surface.

### Example 17

Onto a catheter balloon coated in its folds via the capillary method with paclitaxel which has an amount of 1-2 μg paclitaxel per mm² fold a titanium stent is crimped which is coated with a polymeric carrier system of a polyether sulfone containing the active agent paclitaxel in a preferably cytostatic dosage. The titanium stent was previously coated with a solution of paclitaxel and the polyether sulfone in methylene chloride via the pipetting method. On the titanium stent there are ca. 0.5 μg paclitaxel per mm² stent surface.

### Example 18

A catheter balloon coated with rapamycin/transport mediator is provided. Now a bioresorbable stent of polylactide is crimped onto this catheter balloon which is coated with a coating of polylactide containing paclitaxel in an amount of ca. 1.0 μg per mm² stent surface.

### Example 19

A non-dilated fold balloon is coated completely with an active agent and an excipient as carrier by means of the described pipetting method.

Therefore 150 mg sirolimus are dissolved in 4.5 ml acetone and mixed with a solution of 100 μl isopropyl myristate in 450 μl ethanol. After applying the solution the fold balloon is dried overnight.

### Example 20

The fold balloon coated according to Example 19 is introduced into a PBS filled silicon tube and therein expanded to nominal pressure for 60 sec.

Subsequently, the sirolimus content remaining on the balloon catheter, the portion dissolved in the PBS buffer, and the content of active agent adhering to the inner surface of the tube are determined after extraction with acetonitrile by means of HPLC measurement:

Determining the sirolimus content after expansion of the fold
balloon by means of HPLC measurement [in %]

| on the fold balloon | in PBS buffer | on the inner surface of the tube |
|---|---|---|
| 35.2% | 17.3% | 47.5% |

### Example 21

Coating of a Catheter with the Thread Drag Method

For preparation of the coating solution 100 mg sirolimus are dissolved in 3.5 ml acetone and mixed with a solution of 2 mg acesulfam K in 500 μl ethanol. When initiating the rotation of the catheter a slight negative pressure is applied upon the balloon so that the folds do not turn over during the rotational movement of the balloon about its own longitudinal axis. Subsequently the balloon is pre-wetted with the wetting solution. Immediately after the coating procedure is carried out. A drop of the coating solution is dragged over the balloon through the dosing needle and the dragging wire welded on until the solvent evaporates to such a degree that a solid coating has formed.

After ending the adjusted overcoatings the catheter keeps on rotating for some seconds. Subsequently the catheter is removed from the device and dried at room temperature.

### Example 22

### Covalent Hemocompatible Coating of Stents:

Non-expanded cleansed stents of medical high quality steel LVM 316 are dipped into a 2% solution of 3-aminopropyltriethoxysilane in an ethanol/water mixture (50/50 (v/v)) for 5 minutes and subsequently dried. Subsequently the stents are washed overnight with demineralised water.

3 mg desulfated and reacetylated heparin is dissolved in 30 ml 0.1 M MES buffer (2-(N-morpholino)ethane sulfonic acid) at pH 4.75 and then 30 mg N-cyclohexyl-N′-(2-morpholinoethyl)carbodiimide-methyl-p-toluene sulfonate are added. The stents are stirred in this solution overnight at 4° C. Subsequently intensive rinsing with water and 4M NaCl solution is carried out.

### Example 23

The cleansed or covalently coated stents are crimped onto the balloon catheter and coated together with a spray solution containing an active agent according to example 1A-V by means of the thread drag method.

### Example 24

Coating of the hemocompatibly furnished stents with a matrix loaded with active agent by means of the roll method

Coating solution: a polylactide RG5032/taxol solution of 145.2 mg polylactide and 48.4 mg taxol are filled up to 22 g with chloroform.

### Example 25

Coating of the total system stent+balloon with a matrix loaded with active agent as base coating and active agent as top coating.

Base coating: 19.8 mg linseed oil and 6.6 mg taxol are filled up to 3 g with chloroform

Top coating: 8.8 mg taxol are filled up to 2 g with chloroform.

The balloon catheter with a crimped stent thereon is coated with the base coat by means of the drop-drag method. As soon as this base coat becomes a high viscous film by evaporation of the solvent on the system surface the second layer with the pure active agent can be sprayed on.

### Example 26

Coating of a balloon catheter with a cell membrane affine matrix containing an active agent.

The balloon catheter is mounted by means of an adapter onto the drive shaft of a rotation motor and fixed in such a way that it happens to lie in a horizontal position without bending. After applying a slight negative pressure on the balloon the balloon is coated with the solution according to the set number of balloon tracings.

### Coating Solution:

The transport mediator carrageenan, phosphatidylcholine and glycerol (1:2:2) are dissolved in ethanol/water (1:1; v:v). In 10 ml of this solution subsequently 200 μg Biolimus A9 are dissolved.

### Thread Drag Method:

A drop of the coating solution is dragged over the rotating balloon through the dosing needle and the drag wire welded on until the solvent is evaporated to such a degree that a solid coating has formed. Subsequently, the catheter is removed from the device and dried overnight at room temperature upon further rotation.

### Example 27

A solution of rapamycin in ethanol is prepared and this solution is sprayed twice onto a catheter balloon without a stent and the catheter balloon is dried inbetween by evaporation of the solvent.

After repeating the spray coating twice solution P of example 1 is used in a third step with the transport mediator fosferol as spraying solution, and the catheter balloon is dried for a last time, and an uncoated stent made of metal is crimped onto the balloon. In the spraying solution the ration of rapamycin to fosferol is 10:1.

### Example 28

A thin viscous ethanolic solution of paclitaxel in vanillin is prepared in a ration 2:1, which is thin viscous in such a way that the solution is dragging itself into the folds of the fold balloon by capillary forces. By use of a capillary which is set to one end of the fold the alcoholic paclitaxel solution is flowing into the fold until the inner space of the cavity is filled completely due to capillary forces. The content of the fold is left to dry, the balloon is rotated and the next fold is filled. Each fold is filled only once.

### Example 29

A fold balloon expanded to nominal pressure is dipped into a 1% paclitaxel/chloroform dipping solution with maltol (0.5 weight-%) for 5-10 s and then dried under rotation around the longitudinal axis to such a degree that the major part of the chloroform has evaporated. Before the drying is completed the balloon is deflated again in an air stream. Optionally a further transport mediator can be added to the paclitaxel solution.

### Example 30

The fold balloon is tethered in a horizontal position on the rotatable axis so that the fold to be filled happens to always lie upside. Thus step by step each fold is filled with a solution of a honey- to syrup-like viscosity (viscosities from 10² to 10⁵ mPa·s) of rapamycin in THF with diethylene glycol lauryl ether by means of a teflon cannula being slowly led through from the beginning to the end of the fold as extension of a needle syringe.

For this purpose the teflon cannula is led to the centre of the cavity formed by the fold, and during the movement of the horizontally tethered catheter in its longitudinal direction a defined amount of the high viscous solution is released into the fold cavity (squirting method). The amount of the filled material is limited in such a way that the fold doesn't lift from the balloon body after filling and vary corresponding to different balloon dimensions and manufacturers.

### Example 31

A commercially available catheter balloon is coated with an amount of 3 μg paclitaxel per mm² balloon surface. The coating is carried out via the pipetting method by use of a solution of paclitaxel in acetone and ferulic acid (solution F of example 1). An uncoated metal stent made of cobalt-chromium is then crimped onto the coated catheter balloon.

### Example 32

Paclitaxel is dissolved in DMSO containing ca. 10 vol. % water. Potassium oxalate, sodium chloride, glutaminic acid and oxalic acid and the transport mediator octyl phenol ethoxylate are added to this solution and the catheter balloon is coated several times with this solution by using the thread drag method and dried after every the coating sequence. Subsequently, the coated catheter balloon is provided with a biodegradable layer of a lactam.

### Example 33

A non-dilated fold balloon is coated by means of the described pipetting method completely with an active agent and a transport mediator.

For this purpose 160 mg paclitaxel are dissolved in 5 ml methanol and mixed with a solution of 200 μg 1,2,3-butanetriol in 400 μl ethanol. After application of the solution the fold balloon is dried overnight in a drying cabinet at 70° C.

### Example 34

The balloon catheter is mounted by means of an adapter onto the drive shaft of a rotation motor and tethered in such a way that it happens to lie in a horizontal position without bending. After applying a slight negative pressure on the balloon the balloon is coated with the solution with the set number of 4 balloon tracings. By means of the dosing needle and the drag wire being welded on a drop of the coating solution is dragged over the rotating balloon until the solvent is evaporated to such a degree that a solid coating has formed. Subsequently the catheter is taken off the machine and dried at room temperature and upon further rotation overnight.

### Used Coating Solution:

The transport mediators stearyl alcohol and 1,2,4-butanetriol (1:1, w/w) are dissolved in ethanol/water (3:1; v:v). Subsequently 400 μg paclitaxel are dissolved in 10 ml of this solution.

### Example 35

A commercially available balloon catheter with an expandable balloon of polyamide is provided.

Paclitaxel is dissolved in acetone together with benzethonium fluoride in the concentration of 50 mg paclitaxel and 100 μg benzethonium fluoride per ml acetone. This coating solution is applied onto the catheter balloon by the inventive ballpoint method. The coating formed that way is dried overnight at room temperature and sterilized with ethylene oxide.

### Example 36

Paclitaxel is mixed with magnesium sulphate and sodium acetate and processed by addition of methanol and lanolin to a paste, which is then filled into a syringe and squirted under the folds of a fold balloon. Upon coating the outlet of the squirting nozzle is going along the fold and lays a layer of paste into the fold along the longitudinal direction of the fold. This results in a coating with an amount of 3 μg paclitaxel per mm² balloon surface.

### Example 37

A commercially available balloon catheter is coated with an amount of 2.5 μg picrolimus per mm² balloon surface. This coating is carried out by means of inventive pipetting methods by use of a solution of picrolimus and tetradecyltrimethylammonium chloride in acetone.

### Example 38

A fold balloon expanded to nominal pressure is dipped into a 2% paclitaxel/methanol dipping solution with laurocapram (0.2%) for 10-15 s and is subsequently dried upon rotation around the longitudinal axis to such a degree that the major part of the methanol has evaporated. Before the drying is completed the balloon is deflated again in an air stream.

### Example 39

A non-dilated fold balloon is coated completely via the described pipetting method with an active agent and a transport mediator.

For this purpose 160 mg paclitaxel and 10 mg phenylboronic acid are dissolved in 1 ml methanol. After application of the solution the fold balloon is dried overnight at room temperature. Then the coated catheter balloon is provided with a biologically degradable layer of a lactame.

### Example 40

A fold balloon with three folds is tethered in a horizontal position on the rotatable axis so that the fold to be filled always happens to lie upside. Thus step by step each fold is filled with a solution of a honey- to syrup-like viscosity (viscosities from 10² to 10⁵ mPa·s) of 2.5% of everolimus in acetone with 1 volume percent QUAB 151 by means of a teflon cannula slowly led through from the beginning to the end of the fold as extension of a needle syringe.

For this purpose the teflon cannula is led to the centre of the cavity formed by the fold, and during the movement of the horizontally tethered catheter in its longitudinal direction a defined amount of the high viscous solution is released into the fold cavity (squirting method). The amount of the filled material is limited in such a way that the fold doesn't lift from the balloon body after filling and vary corresponding to different balloon dimensions and manufacturers. An uncoated metal stent of cobalt-chromium is then crimped onto the coated catheter balloon.

### Example 41

Coating of the total system stent+balloon with a matrix loaded with active agent as a base coating and an active agent as a top coating

Base coating: 19.8 mg linseed oil, 0.3 mg alkyl-(polyoxyethyl) phosphate and 6.6 mg taxol are filled up with chloroform up to 3 g

Top coating: 8.8 mg taxol and 0.5 mg alkyl-(polyoxyethyl)-phosphate are filled up with chloroform up to 2 g.
The balloon catheter with crimped stent is coated with the base coating via the drop drag method. As soon as this base coating becomes a high viscous film by the evaporation of the solvent on the surface of the system, a second layer of active agent can be sprayed on by the inventive method.

### Example 42

A commercially available balloon catheter is coated with an amount of von 2.5 μg paclitaxel per mm² balloon surface. The coating is carried out by the inventive pipetting method by use of a solution of 0.5 mg/ml paclitaxel in squalene.

### Example 43

A solution of paclitaxel in methanol is prepared and this solution is sprayed onto a catheter balloon without a stent for three times and the catheter balloon is dried in between by evaporation of the solvent.

After repeating the spray coating for three times, the solution of paclitaxel with the transport mediator diethyl sulfoxide as a spraying solution is used, and the catheter balloon is dried for a last time and an uncoated stent of metal is crimped onto the balloon. In this spraying solution the ratio of paclitaxel and diethyl sulfoxide is 1:1.

Hitherto performed experiments have shown that the selected transport mediators have a similarly good effect on the used active agents, such as the already published substances urea and citric ester.

### Example 44

42.7 mg (0.05 mmol) paclitaxel are dissolved in 5 ml chloroform and 9.5 mg (0.03 mmol) tetrapropyl tartrate are added.

3 ml coating solution are sprayed onto the catheter balloon in three steps, and the catheter balloon is air-dried after each spraying step for at least 10 minutes.

It could have been shown that such a catheter balloon coated with tetrapropyl tartrate and paxlitaxel accordingly is well suited to transfer the paclitaxel as completely as possible onto the inner wall of the blood vessel during dilation, whereby a good prophylaxis for restenosis can be obtained.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

1-23. (canceled)

24. Balloon catheter containing a catheter balloon with a dried oily or solid coating of at least one pharmacologic agent and a transport mediator or a mixture of transport mediators, wherein the transport mediator or the mixture of transport mediators has a boiling point of at least 150° C., the transport mediator or the mixture of transport mediators has an oily or solid consistency at 20° C. and causes no immune reaction, for coating of catheter balloons for vessel dilatation, wherein the transport mediator or the mixture of transport mediators is not a contrast agent.

25. Balloon catheter according to claim 24, wherein the transport mediator is not a polymer and the mixture of transport mediators does not contain a polymer.

26. Balloon catheter according claim 24, wherein the transport mediator has at least 6 carbon atoms or at least two oxygen atoms or at least one nitrogen atom.

27. Balloon catheter according claim 24, wherein the transport mediator or the mixture of transport mediators has a vapor pressure of less than 25 kPa at 20° C.

28. Balloon catheter according to claim 24, wherein the transport mediator is lipophilic and has a partition coefficient between butanol and water of ≧0.5.

29. Balloon catheter according to claim 24, wherein the transport mediator is lipophilic and hydrophilically esterified in such a way that the transport mediator has a partition coefficient between butanol and water of ≦0.5.

30. Balloon catheter according to claim 24, wherein the transport mediator is hydrophilic and hydrophobically esterified in such a way that the transport mediator has a partition coefficient between butanol and water of ≧0.5.

31. Balloon catheter according to claim 24, wherein the transport mediator or the mixture of transport mediators does not form micelles which are hydrophilic to the outside.

32. Balloon catheter according to claim 24, wherein the transport mediator or the mixture of transport mediators is pH neutral.

33. Balloon catheter according to claim 24, wherein the transport mediator or the mixture of transport mediators has a pH value of 9>pH>7 in aqueous solution.

34. Balloon catheter according claim 24, wherein the transport mediator or the mixture of transport mediators has a pH value of 5<pH<7 in aqueous solution.

35. Balloon catheter according to claim 24, wherein the transport mediator has at least one ionic or ionizable functional group.

36. Balloon catheter according claim 24, wherein the transport mediator is able to form hydrogen bonds.

37. Balloon catheter according claim 24, wherein the transport mediator is able to increase the moisture of the cell wall.

38. Balloon catheter according to claim 24, wherein the transport mediator is able to cleave the hydrogen bonds in the cell wall.

39. Balloon catheter according claim 24, wherein the transport mediator can interact with the lipids of the lipid bilayer and/or with the hydrocarbons of the lipid bilayer.

40. Balloon catheter according to claim 24, wherein the transport mediator has a molecular weight of 150 g/mol to 300 g/mol.

41. Balloon catheter according to claim 24, wherein at most 50% by weight of the transport mediator or the mixture of transport mediators is volatilized at 25° C. after 2 months.

42. Balloon catheter according claim 24, wherein the transport mediator is able to pass trough the plasma membrane.

43. (canceled)

44. Balloon catheter according to claim 24, wherein the transport mediator is selected from the group consisting of: amides, phenols, phenolic esters, phenolic ethers, aromatic alcohols, aromatic acids, sulfoxides, organic boron compounds, polyvalent alcohols with 2 to 6 carbon atoms, monoglycerides of fatty acids and alcohols, fatty acid ethers, terpene hydrocarbons, alcohols with at least 8 carbon atoms, heterocyclic compounds, alkaloids, nanoparticles, enzymes and quaternary ammonium salts.

45. Balloon catheter according to claim 44, wherein the amides, phenols, phenolic esters, phenolic ethers, aromatic alcohols, aromatic acids, sulfoxides, organic boron compounds, polyvalent alcohols with 2 to 6 carbon atoms, monoglycerides of fatty acids and alcohols, fatty acid ethers, terpene hydrocarbons, alcohols with at least 8 carbon atoms, heterocyclic compounds, alkaloids, nanoparticles, enzymes and quaternary ammonium salts are selected from: urea, DMF, DMA, cyclophosphamide, alkanolamides, anisole, anethole, vanillin, coniferin, thymol, carvacol, salicylic acid, salicylic alcohol, phenylethanol, caffeic acid, ferulic acid, cinnamyl alcohol, adrenaline, dopamine, amphetamine, boric acid ester, 1,2 ethanediol, 1,2 propanediol, 1,3 propanediol, propanetriol, lactitol, mannitol, dulcitol, isomalt, sucrose, xylitol, alitame, maltitol, 2-ethyl-1,3-hexanediol, glycerine monooleate, glycerine monolinoleate, glycerine monolaurate, maltol, meglumine, acyl glyceride, polyoxyethylene lauryl ether, diethylene glycol lauryl ether, polyethylene glycol monolauryl carboxymethyl ether, monocyclic terpenes, thymol, α-terpineols, β-terpineols, gamma-terpineols, 1,8-terpin, 1,8-cineol, bicyclic terpenes, caranes, pinanes, bornanes, α-pinene, 3-carene, camphene, borneol, camphor, monocyclic sesquiterpenes, bisabolene, farnesol, acyclic terpenes, myrcene, ocimene, linalool, tricyclic sesquiterpenes, santalene, triterpenes, squalenoids, squalene, fusides, tetracyclic triterpene acid, lanosterol, tetraterpenes, carotinoids, carotene, lycopene, lutein, zeaxanthin, crocetin, lipochromes, polyprenes, male and female steroid hormones, androgens, estrogens, gestagens, testosterone, androsterone, estriol, estradiol, estrone, fosfestrol, prolutone, progesterone, corticoids, cortisol, cortisone, aldosterone, triamcinolone, alcanols, myristyl alcohol, stearly alcohol, sterols, alkyl-2-(N,N-disubstituted amino)-alkanoates, alkyl-2-(N,N-disubstituted amino)-alkanol alkanoates, N-methylpyrrolidone, bilirubin, biotin, sulfamethoxazole, 1-substituted azacylcloalkan-2-one, laurocapram, (1-dodecylazacycloheptan-2-one) and derivatives, cyclodextrins, azacyclo alkenes, chlorhydrin, glycidyl trimethylammonium halogenide, 3-chloro-2-hydroxypropyltrimethylammonium halogenide, dodecyltrimethylammonium halogenide, hexadecyltrimethyl-ammonium halogenide, tetradecyltrimethylammonium halogenide, sodium stearyl fumarate, fumaric acid and alkyl-(polyoxyethyl)-phosphate, carrageenan.
